(19) United States
(12) Patent Application Publication
(10) Pub. No.: US 2011/0218488 A1
(43) Pub. Date: Sep. 8, 2011

(54) Systems and Methods for Implanting Devices in the Bladder and Other Genitourinary Sites

(75) Inventors: Jim Boyko, Attleboro, MA (US); Heejin Lee, Arlington, MA (US); Cheryl Larrivee-Elkins, Framingham, MA (US)

(73) Assignee: TARIS BIOMEDICAL, INC., Lexington, MA (US)

(21) Appl. No.: 13/042,200

(22) Filed: Mar. 7, 2011

Related U.S. Application Data
(60) Provisional application No. 61/311,103, filed on Mar. 5, 2010.

(51) Int. Cl. A61M 31/00

(52) U.S. Cl. 604/57

(57) ABSTRACT

An implantable drug delivery device includes a drug reservoir portion, a retention frame portion, and a guide wire portion. The drug reservoir portion houses a drug. The retention frame portion houses a retention frame. The guide wire portion defines a hollow bore for receiving a guide wire.

### CROSS-REFERENCE TO RELATED APPLICATIONS

The present application claims priority to U.S. Provisional Application No. 61/311,103, which was filed Mar. 5, 2010, which is incorporated herein by reference in its entirety.

### BACKGROUND

The present disclosure generally relates to systems and methods for implanting devices in the bladder and other genitourinary sites, particularly with reference to catheters and stylets for deploying implantable drug delivery devices in the bladder.

A catheter is a device that is positioned in the body to provide ingress and egress to sites within the body. Often, catheters are employed to provide ingress and egress to genitourinary sites within the body, such as the bladder or other locations within urological or reproductive systems. However, known catheters are not suited for deploying implantable devices into the bladder. Some known catheters are designed for the instillation or drainage of fluid, but these catheters often have narrow interior lumens that are not wide enough for passage of an implantable device. Further, most known catheters are not suited for deploying devices that are flexible. Particularly, flexible devices may not be able to exit a distal end of many known catheters without getting lodged or wedged in an exit opening.

A need therefore exists for systems and methods for deploying devices to genitourinary sites in the body. More particularly, a need exists for systems and methods for deploying flexible drug delivery devices into the bladder.

### SUMMARY

An implantable drug delivery device includes a drug reservoir portion, a retention frame portion, and a guide wire portion. The drug reservoir portion houses a drug formulation. The retention frame portion houses a retention frame. The guide wire portion defines a hollow bore for receiving a guide wire.

In embodiments, the drug formulation may include a plurality of drug tablets. At least some of the drug tablets may include lidocaine. The retention frame may include an elastic wire. The retention frame may be configured to assume a pretzel-like shape. The device may be deformable from a retention shape to an elongated shape.

Another implantable drug delivery device includes a device body that has one or more walls. The walls define a drug reservoir lumen, a retention frame lumen, and a guide wire lumen. A plurality of solid drug tablets are housed within the drug reservoir lumen, a retention frame is housed within the retention frame lumen, and a hollow bore is within the guide wire lumen.

In embodiments, the device body may be deformable and water-permeable. At least some of the solid drug tablets may include lidocaine. An interstice may be formed between any two adjacent drug tablets, the interstice facilitating deformation of the device body. The device body may be integrally formed by an extrusion process or a molding process. The device body may include a water permeable silicone. The retention frame may be configured to assume a pretzel-like shape. The retention frame may include an elastic wire. The hollow bore may be sized and shaped for receiving a guide wire.

A system includes an implantable drug delivery device and a guide wire deployment system for implanting the implantable drug delivery device. The implantable drug delivery device includes a device body that has one or more walls, the walls defining a drug reservoir lumen, a retention frame lumen, and a guide wire lumen. A plurality of solid drug tablets are housed within the drug reservoir lumen, a retention frame is housed within the retention frame lumen, and a hollow bore is housed within the guide wire lumen. The guide wire deployment system includes a guide wire and a plunger device for pushing the implantable drug delivery device over the guide wire.

In embodiments, the guide wire may be long enough to extend through the urethra to the bladder. The guide wire may have a cross-sectional area that is sized and shaped for fitting through the hollow bore of the implantable drug delivery device. The plunger device may include a plunger, a handle, a sheath, and an internal bore. The sheath may extend between the plunger and the handle for transferring to the plunger a driving force applied to the handle. The internal bore may receive the guide wire, such that the plunger device can travel over the guide wire. The pusher may include a stop configured to indicate that the implantable drug delivery device has separated from the guide wire.

### BRIEF DESCRIPTION OF THE DRAWINGS

FIG. 1 is a plan view of an embodiment of a drug delivery device.

FIG. 2 is a cross-sectional plan view of the drug delivery device shown in FIG. 1, illustrating the drug delivery device inside a deployment instrument.

FIG. 3 is a cross-sectional view of the drug delivery device shown in FIG. 1, taken along line 3-3.

FIG. 4 is a plan view of a portion of a deployment instrument, illustrating the drug delivery device exiting a distal end of the deployment instrument.

FIG. 5 is a sagittal view of a male genitourinary system, illustrating the drug delivery device being deployed through the urethra into the bladder.

FIG. 6 is a plan view of an embodiment of a deployment instrument.

FIG. 7 is a cross-sectional plan view of a proximal end portion of the embodiment of a deployment instrument shown in FIG. 6.

FIG. 8 is a cross-sectional plan view of a distal end portion of the embodiment of a deployment instrument shown in FIG. 6.

FIG. 9 is a cross-sectional plan view of another embodiment of a distal end portion for a deployment instrument.

FIG. 10 is a cross-sectional plan view of an additional embodiment of a distal end portion for a deployment instrument.

FIG. 11 is a plan view of a further embodiment of a distal end portion for a deployment instrument.

FIG. 12 is a plan view of yet another embodiment of a distal end portion for a deployment instrument.

FIG. 13 is a plan view of an embodiment of a stylet.

FIG. 14 is a cross-sectional plan view of a distal end portion of the stylet shown in FIG. 13.

FIG. 15 is a cross-sectional plan view of a proximal end portion of the stylet shown in FIG. 13.

FIG. 16 is a plan view of an embodiment of a distal tip of a stylet.

FIG. 17 is a plan view of another embodiment of a distal tip of a stylet.

FIG. 18 is a plan view of an additional embodiment of a distal tip of a stylet.

FIG. 19 is a cross-sectional plan view of an embodiment of a stylet associated with a deployment instrument.

FIG. 20 is a cross-sectional plan view of another embodiment of a stylet associated with a deployment instrument.

FIG. 21 is a cross-sectional plan view of another embodiment of a deployment instrument.

FIG. 22 is a cross-sectional plan view of an additional embodiment of a deployment instrument.

FIG. 23 is a cross-sectional plan view of a further embodiment of a deployment instrument.

FIG. 24 is a perspective view of an embodiment of a deployment instrument.

FIG. 25 is a perspective view of a distal end portion of the deployment instrument shown in FIG. 24.

FIG. 26 is another perspective view of the distal end portion of the deployment instrument shown in FIG. 24, illustrating an implantable device exiting the deployment instrument.

FIG. 27 is a cross-sectional plan view of an embodiment of a transfer plug associated with an implantable device.

FIG. 28 is a cross-sectional plan view of another embodiment of a transfer plug associated with an implantable device.

FIG. 29 is a cross-sectional plan view of an embodiment of a transfer plug positioned adjacent to an implantable device.

FIG. 30 is a perspective view of another embodiment of a deployment instrument.

FIG. 31 is a perspective view of a distal end portion of the deployment instrument shown in FIG. 30, illustrating an implantable device exiting the deployment instrument.

FIG. 32 is a perspective view of another embodiment of a deployment instrument for deploying an implantable device, wherein FIG. 32A is a partial cut-away perspective view of the deployment instrument, illustrating the implantable device exiting the deployment instrument as a handle is actuated, FIG. 32B is a partial cut-away, perspective view of a distal end of the deployment instrument, illustrating an implantable device within the deployment instrument, and

FIG. 32C is a partial cut-away, perspective view of a distal end of the deployment instrument illustrating the implantable device exiting the deployment instrument.

FIG. 33 illustrates a further embodiment of a deployment system for deploying an implantable drug delivery device into a bladder, wherein FIG. 33A is a partial cut-away, perspective view of the deployment instrument, and FIG. 33B is a partial cut-away, perspective view of a distal end of the deployment instrument, FIG. 33C is a partial cut-away, perspective view of the deployment instrument illustrating the handle being actuated to deploy the implantable device, and FIG. 33D is a partial cut-away, perspective view of a distal end of the deployment instrument, illustrating the implantable device exiting the catheter as the handle is actuated.

FIG. 34 illustrates yet another embodiment of a deployment instrument for deploying an implantable drug delivery device into a bladder, wherein FIG. 34A is a perspective view of the deployment instrument, FIG. 34B is a partial cut-away, perspective view of a distal end of the deployment instrument, and FIG. 34C is a partial cut-away, perspective view of a portion of the deployment instrument, illustrating strings within channels in its wall.

FIG. 35 illustrates an embodiment of an implantable device designed to be implanted over a guide wire, wherein FIG. 35A is a plan view of the implantable device and FIG. 35B is a cross-sectional view of the device taken along line 35B-35B in FIG. 35A.

FIG. 36 illustrates an embodiment of a guide wire deployment system for deploying an implantable device into a bladder, wherein FIG. 36A is a perspective view of the deployment system before the implantable device is implanted, and FIG. 36B is a perspective of the deployment system as the implantable device is implanted.

FIG. 37 is a block diagram of an embodiment of a method of implanting an implantable device in the bladder.

### DETAILED DESCRIPTION

Described below are embodiments of systems and methods for deploying an implantable medical device into an implantation site of a body. In particular embodiments, the systems and methods deploy an implantable medical device through one or more natural lumens of the body, such as the urethra, into a genitourinary site of a body, such as the bladder. The systems and methods facilitate wholly implanting an implantable medical device in the body, and the implanted device may remain in the body after the deployment system has been removed or the deployment method has ended. For example, the implanted device may be a drug delivery device that releases drug into the body over an extended implantation period. The drug delivery device may be implanted in the bladder, free-floating within the bladder and releasing one or more drugs over an extended period.

In some embodiments, a deployment system is provided. The deployment system generally includes a deployment instrument, such as a catheter or other device, that can be used to deploy an implantable device through the urethra into the bladder or another genitourinary site. The deployment instrument may be specially configured for navigating the urethra to the bladder.

In other embodiments, a deployment method is provided. The deployment method may include deploying an implantable device through a deployment instrument positioned in a natural lumen of the body, such as the urethra, into a body cavity such as the bladder. The implantable device may be driven through the deployment instrument using a flow of fluid or a stylet. The deployment instrument subsequently may be removed from the body, leaving the implantable device wholly implanted in the bladder or other genitourinary site. For the purposes of this disclosure, the term genitourinary site refers to any location within a urological or reproductive system of the body, such as a bladder, kidney, urethra, ureter, penis, testicle, seminal vesicle, vas deferens, ejaculatory duct, prostate, vagina, uterus, ovary, or fallopian tube, among others or combinations thereof. However, the implantation site may be any location in the body of a human or other mammal, whether male or female, adult or child.

The deployment systems and methods can be used to implant a wide range of medical devices into the body. In a preferred embodiment, the implantable device is a drug delivery device suited for releasing one or more drugs into the body. In particular embodiments, the drug delivery device may deliver lidocaine or another anesthetic or analgesic agent locally to the bladder over a relatively extended period for the treatment of a condition such as IC/PBS, neurogenic bladder, or pain such as post-operative pain.

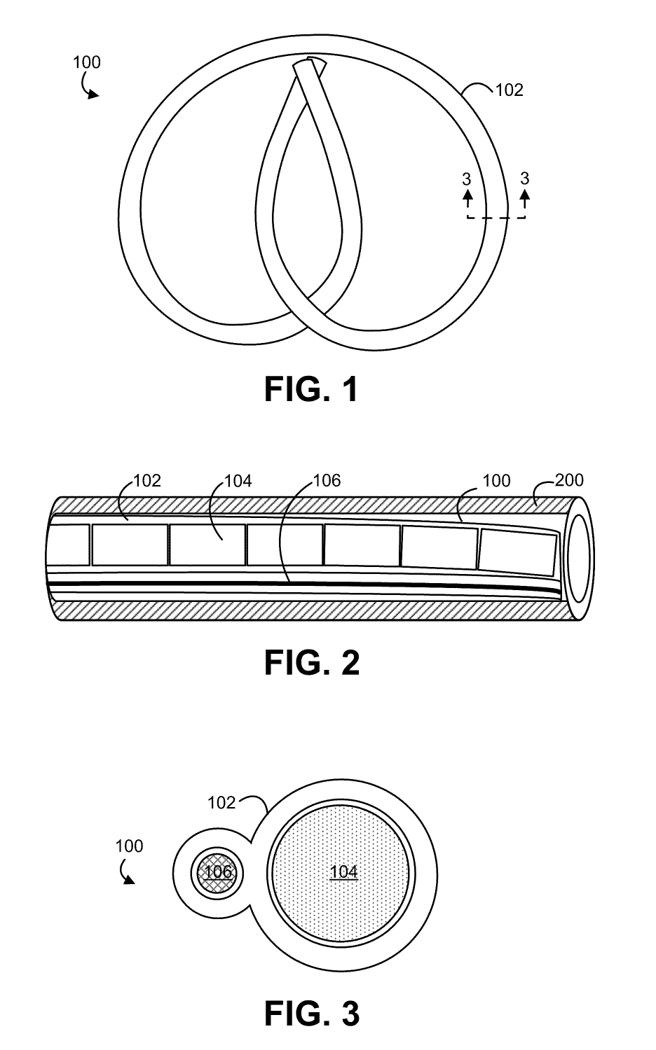

One example of such a drug delivery device 100 is shown in FIG. 1, which is designed for implantation in the bladder. In particular, the drug delivery device 100 generally has a retention shape suited for retaining the device in the body, such as in the bladder. The retention shape may be the illustrated pretzel shape, although other shapes, e.g., overlapping coiled configurations, can be used. The device 100 also has a flexibility that permits deforming the device 100 into a deployment shape suited for inserting the device 100 through a deployment instrument. For example, the device 100 may assume an elongated or linear shape, such as an uncoiled version of the pretzel shape, when positioned within a working channel of a deployment instrument 200, as shown in FIG. 2.

As shown in FIG. 3, which is a cross-sectional view of the drug delivery device 100 taken along line 3-3 in FIG. 1, the illustrated device 100 has a device body 102 that defines two internal lumens, which are aligned along their length. One of the lumens houses a drug payload 104, while the other lumen houses a retention frame 106. The walls of the device body 102, which define the lumens, are formed from flexible and/or elastic materials, such as silicone. The retention frame 106 is likewise formed from flexible and/or elastic materials, such as nitinol. The drug payload 104 also may be in a relatively flexible form, such as in the form of a number of discrete solid drug tablets having breaks or interstices formed between them, as shown in FIG. 2. Thus, the device 100 has a flexibility that permits deforming the device between a retention shape as shown in FIG. 1 and a deployment shape as shown in FIG. 2.

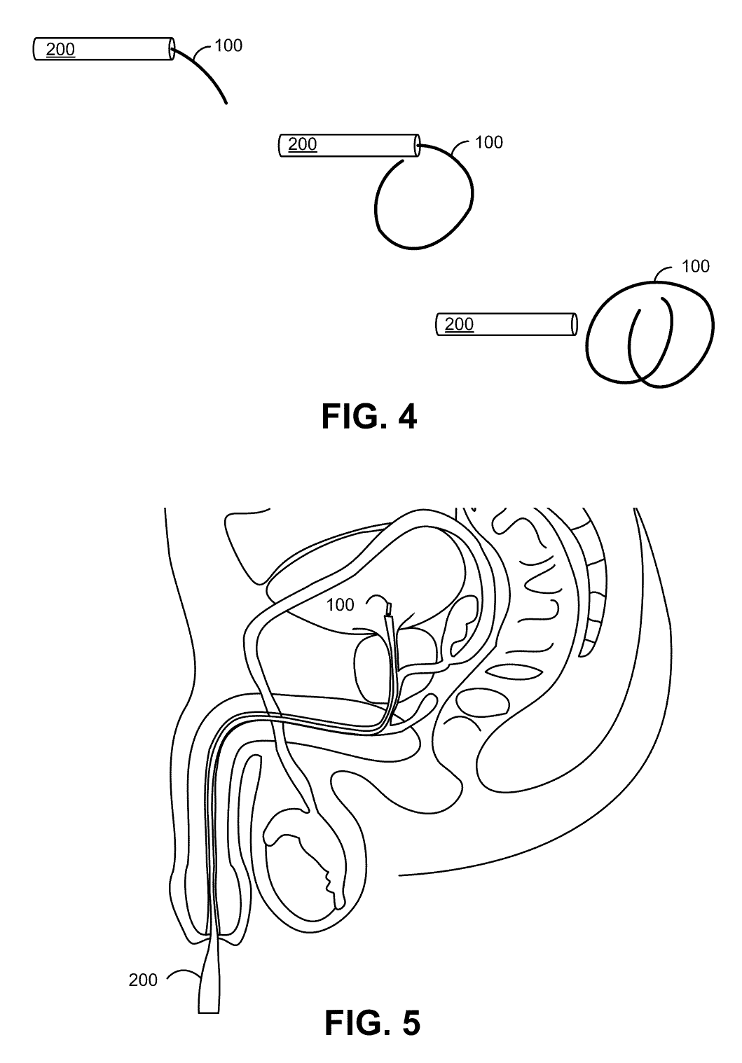

In some embodiments, the device 100 naturally assumes the retention shape, but can be deformed into the deployment shape for implantation. Once in the deployment shape, the device can be passed through the deployment instrument 200. The walls of the deployment instrument 200 exert a force on the device 100 that retains the device in the deployment shape. As the device 100 exits the deployment instrument 200, the force is removed and the device 100 returns to the retention shape for retention in the body. An example is shown in FIG. 4, which illustrates the device 100 returning to the retention shape as the device is deployed from the deployment instrument 200. However, the device may be manually adjusted between the retention shape and the deployment shape, such as through the use of a medical instrument.

Additional embodiments of this and other drug delivery devices are described in the following U.S. patent applications, which are incorporated by reference herein: U.S. application Ser. No. 11/463,956, filed Aug. 11, 2006; U.S. application Ser. No. 12/333,182, filed Dec. 11, 2008; U.S. application Ser. No. 12/538,580, filed Aug. 10, 2009; U.S. application Ser. No. 12/825,215, filed Jun. 28, 2010; U.S. application Ser. No. 12/825,238, filed Jun. 28, 2010; U.S. application Ser. No. 12/851,494, filed Aug. 5, 2010; U.S. application Ser. No. 12/870,261, filed Aug. 27, 2010; U.S. application Ser. No. 12/879,638, filed Sep. 10, 2010; U.S. application Ser. No. 12/963,621, filed Dec. 8, 2010; U.S. application Ser. No. 12/972,364, filed Dec. 17, 2010; U.S. Provisional Application No. 61/370,902, filed Aug. 5, 2010; U.S. Provisional Application No. 61/371,139, filed Aug. 5, 2010; U.S. Provisional Application No. 61/390,495, filed Oct. 6, 2010; U.S. Provisional Application No. 61/390,549, filed Oct. 6, 2010; U.S. Provisional Application No. 61/405,379, filed Oct. 21, 2010; U.S. Provisional Application No. 61/431,334, filed Jan. 10, 2010; U.S. Provisional Application No. 61/439,665, filed Feb. 4, 2011; and U.S. Provisional Application No. 61/444,278, filed Feb. 18, 2011.

The deployment systems and methods described below can be used to deliver the drug delivery device 100 into a genitourinary site of the body, such as the bladder. An example is shown in FIG. 5, which illustrates the device 100 being deployed from the deployment instrument 200 into the bladder. The deployment instrument 200 is sized and shaped for passing through the urethra to the bladder as shown in FIG. 5, wherein the male anatomy is shown by way of example. The drug delivery device 100 can be released from the deployment instrument 200 into the bladder, and once implanted the device 100 releases drug into the bladder for an extended period. However, the deployment systems and methods disclosed herein can be used to implant other devices into the body, or alternatively, to provide access into the body for reasons other than the implantation of a device.

Embodiments of deployment systems and methods are shown and described below. Generally, the deployment systems include a deployment instrument that is designed to navigate natural lumens of the body to reach an implantation site. The deployment instrument is relatively flexible, which permits inserting the instrument through tortuous pathways of the body to the implantation site, and yet the deployment instrument has sufficient column strength to impede or prevent buckling as the deployment instrument is pushed forward. In some cases, the configuration of the deployment instrument may vary to compensate for differences between male and female anatomy. For example, the instrument may be longer and more compliant for use with male patients, or shorter and semi-rigid for use with female patients. In particular embodiments, the deployment instrument may be a catheter or a portion of another medical device.

In embodiments, the deployment instrument generally includes a central body portion, a proximal end portion, and a distal end portion. The central body portion is configured for extending through natural lumens of the body to the implantation site. For example, the central body portion may be sized and shaped for passing through the urethra to the bladder. The distal end portion is configured for traversing the natural lumens to reach the implantation site. In particular, the distal end portion is strong enough to permit driving the deployment instrument through the natural lumens of the body without buckling or jamming, and yet is flexible enough to minimize or avoid damaging the tissue lumen walls. For example, the distal end portion may traverse the urethra with no or minimal damage to its walls. The proximal end portion remains outside of the body, providing access for a user, such as a medical professional, or an interface for equipment, such as medical equipment. The deployment instrument also includes at least one internal passageway or lumen extending along its longitudinal length that permits passing a device through the interior of the instrument, so that the device is protected during the implantation procedure.

In addition to permitting passage of the device, the internal passageway may serve other purposes. In some embodiments, the internal passageway may provide a path for fluid to travel through the instrument. For example, lubricant or another suitable fluid may be driven through the internal passageway to drive the device from the internal passageway into the body. As another example, water or another suitable fluid may be delivered through the internal passageway before the device is implanted to form a cushion in the bladder for receiving the device, impeding the device from contacting the bladder wall once implanted. Water also may be directed through the internal passageway to a Foley balloon on a distal end of the instrument for the purpose of inflating the Foley balloon. The internal passageway also may return urine from the bladder to verify the instrument has reached the bladder. In some cases, the deployment instrument includes mechanics operable to eject the drug delivery device from the instrument or to retrieve a previously deployed device back into the instrument, and these mechanics may be housed in the internal passageway. Any number of internal passageways or lumens can be provided, with each lumen being useful for one or a combination of the purposes described above. For example, the deployment instrument may include a main lumen that is used to deliver the drug delivery device into the bladder, a smaller urine return lumen that is used to verify placement of the deployment instrument distal tip in the bladder, and a separate Foley balloon inflation lumen that is in direct fluid communication with the interior of a Foley balloon.

Each internal passageway may extend from a proximal opening on the proximal end portion of the deployment instrument to an distal opening on the distal end portion for the deployment instrument. The distal openings may have various placements or sizes along the distal end portion depending on their purpose, as described below. For example, a urine return opening in communication with a urine return lumen may be spaced downward from the distal-most tip of the deployment instrument by a selected distance, so that urine only passes into the urine return lumen once the distal tip of the deployment instrument is positioned at least the selected distance in the bladder.

At least one exit opening in communication with the internal passageway permits releasing the device from the internal passageway into the bladder or other implantation site. In embodiments, the exit opening is configured to facilitate smooth passage of the device from the interior passageway through the exit opening.

In embodiments, the distal end portion includes one or more of a coude tip, a council tip, and an inflatable balloon. A coude tip may be useful for traversing tortuous pathways through the anatomy, such as near the prostate gland in male patients. A council tip may be useful for inserting the catheter over a guidewire. An inflatable balloon may be inflated to retain the distal end portion of the catheter in the bladder. These distal tip configurations can be used alone or in combination. For example, the use of both a coude tip and inflatable balloon may be especially useful with male patients due to the structure of the male anatomy.

In some embodiments, the proximal end portion has a larger diameter or cross-sectional area than other portions of the deployment instrument to facilitate loading an implantable device into the instrument. The proximal end portion also may be formed from a relatively stiffer material, while the central body and distal portions may be relatively more compliant for navigating through the urethra. The proximal end portion may be associated with a handle that is grasped by the user to hold the deployment instrument. In some embodiments, the handle can be actuated by a user to eject the drug delivery device from the catheter, as described below. In other embodiments, the proximal end portion is associated with a luer connector that facilitates forming a relatively water-tight connection between the deployment instrument and an external fluid source, such as a syringe, to introduce fluid into the deployment instrument. The fluid may drive the device toward the implantation site or the fluid may form a cushion in the implantation site for receiving the device, as described below.

The proximal end portion may also have one or more ports, each of which may be in communication with one or more internal passageways or lumens through the deployment instrument. For example, a urine return port may be in communication with a urine return lumen through the catheter, which directs urine from the bladder. As another example, a fluid introduction port may be in communication with a Foley balloon at a distal end of the catheter by way of another separate lumen, so that fluid from outside of the body can be directed to the Foley balloon to inflate it. Also, any one internal passageway may be in communication with more than one port. For example, one internal passageway may be in communication with both a urine return port and a fluid introduction port. In some embodiments, any one of the ports may be associated with a valve that permits or prevents the flow of fluid through the port. For example, a valve on a urine return port may be opened so that urine can return through the port to verify placement of the distal tip in the bladder, and thereafter the valve may be closed to prevent the continued return of urine through the urine return port during the implantation process. Alternatively, one valve may be associated with the deployment instrument upstream of a number of ports, so that fluid flow between the deployment instrument and all of the ports can be controlled by opening or closing one valve. Examples of such configurations are described in further detail below.

The dimensions of the instrument are selected based at least in part on the dimensions of the anatomy that the deployment instrument is designed to navigate. In embodiments in which the instrument extends through the urethra to the bladder, for example, the central body portion may have a length in the range of about 30 cm to about 45 cm and an outer diameter in the range of about 3 mm to about 7 mm. An inner diameter of the central body portion is selected based at least in part on the dimensions of the device that the instrument is designed to implant. For example, the instrument can be used to implant an embodiment of the drug delivery device 100 shown in FIG. 1, in which case the inner diameter of the central body portion may be in the range of about 2 mm to about 5 mm. The outer diameter of the instrument is dimensioned to pass through the urethra. For example, the outer diameter may be in the range of about 12 to 18 Fr. In some embodiments, the outer diameter is selected to compensate for differences in the male and female anatomy, with a male version of the deployment instrument having a relatively wider outer diameter than a female version of the deployment instrument. For example, the male instrument may have an outer diameter in the range of about 14 to 18 Fr, such as about 16 Fr, while the female instrument may have an outer diameter in the range of about 12 to 16 Fr, such as about 14 Fr. The stiffness of the deployment instrument also may vary according to the anatomy that the deployment instrument is designed to navigate. For example, an embodiment of the deployment instrument suited for navigating the tortuous urethra of the male anatomy may have a thicker wall than an embodiment of the deployment instrument suited for navigating the urethra of the female anatomy.

The wall of the instrument may be relatively thin so that the inner diameter of the instrument is relatively large given the outer diameter of the instrument. Thus, the diameter of the lumen through the instrument may be relatively large in view of the overall size of the instrument as a whole. The wall is formed partially or fully from a material that has a column strength suited to prevent buckling despite its thin nature, yet has a flexibility suited for navigating tortuous pathways through natural body lumens such as the urethra. The material also is biocompatible and is compatible with materials used to form the device to be implanted. The material is also amenable to suitable sterilization processes.

One example of a material that can be used for the deployment instrument is medical grade polyvinyl chloride (PVC). The PVC may be stabilized to permit sterilization with gamma irradiation, which may permit sterilizing the deployment instrument and device together. Other suitable materials include polyethylene (PE), polytetrafluoroethylene (PTFE), styrenic thermoplastic elastomer (STE), silicone, latex rubber, polyurethane, Silitek®, C-flex®, Percuflex®, Tecoflex®, or combinations thereof. In embodiments, the material may be a polymer or copolymer whose composition may be modified or properties enhanced to yield the desired column strength. The deployment instrument can be formed by extrusion, injection molding, or other manufacturing processes known in the art.

The deployment instrument may have markings along its length, such as graduated markings that indicate dimensions or relative positions. The markings may facilitate deploying the deployment instrument in the urethra and determining when the deployment instrument has become properly positioned therein. However, the markings are not necessary.

The implantable device can be loaded into the deployment instrument, through either the exit opening on the distal end portion, through an entry opening on the proximal end portion, or through a slit along the central body portion. The loading location may depend on whether the device is loaded into the deployment instrument before or after the deployment instrument is positioned in the body. The loading location also may depend on how the device is ejected from the deployment instrument into the bladder.

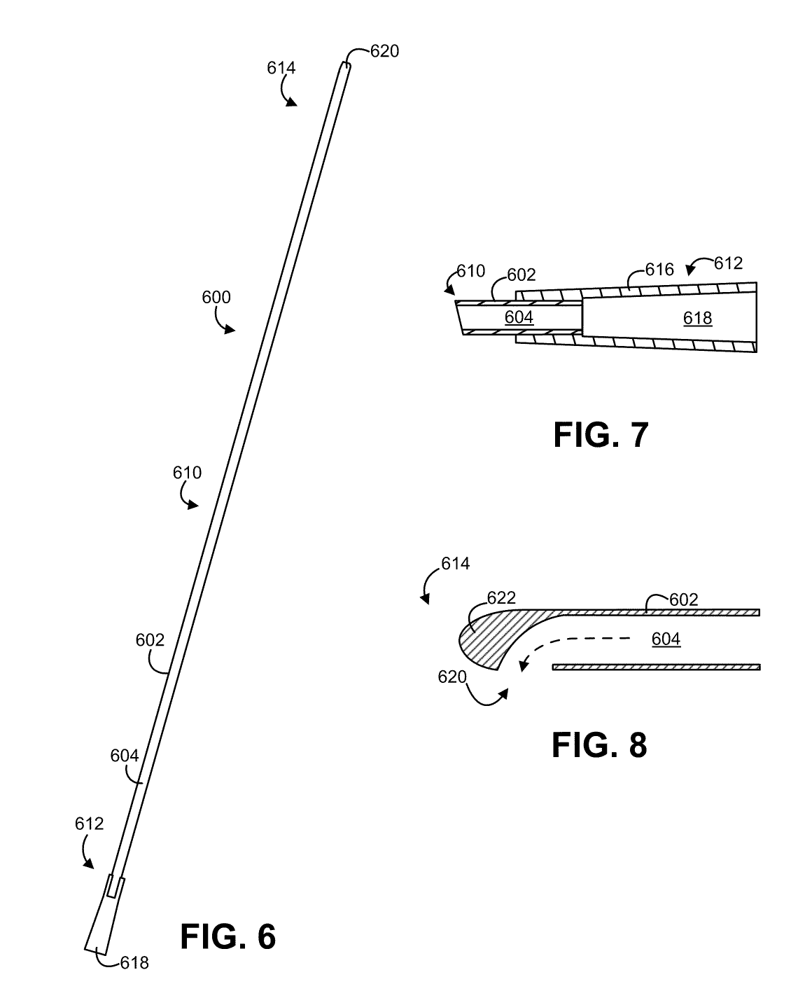

FIG. 6 illustrates an embodiment of a deployment instrument 600 that is suited for implanting a drug delivery device or other implantable device in the bladder. The deployment instrument 600 is generally a catheter or a tube having a wall 602 that defines an interior passageway 604, a portion of which is shown in FIGS. 7 and 8. The interior passageway 604 permits passing a device through the interior of the deployment instrument 600, so that the device is protected during the implantation procedure. The wall 602 of the deployment instrument 600 is relatively thin, so that the interior passageway 604 is relatively wide in view of the overall width of the deployment instrument 600. Thus, a relatively larger device can be inserted through a relatively smaller interior passageway 604. The wall 602 is relatively flexible, which permits inserting the deployment instrument 600 through tortuous pathways of the body to the implantation site, and yet the wall 602 has sufficient column strength to impede or prevent the deployment instrument 600 from buckling as the catheter is pushed forward. In embodiments, at least a portion of the wall 602 can be reinforced to improve the kink resistance and rigidity of the deployment instrument 600. For example, the wall 602 may have a reinforcing material embedded on at least a portion of its interior or applied about at least a portion of its exterior. The reinforcing material can be a steel wire or ribbon that is braided, wound, or wrapped about the exterior. The wire or ribbon can be encapsulated within a polymer layer that is applied about the exterior of the deployment instrument 600, such as by coating or re-extruding the deployment instrument with the polymer. Other reinforcing materials also can be used. In the illustrated embodiment, the deployment instrument 600 is a hollow cylindrical tube, although other shapes, such as shapes having oval or elliptical cross-sections, are possible. For example, the cross-sectional shape of the tube may match the cross-sectional shape of the implantable device.

More particularly, the deployment instrument 600 includes a central body portion 610, a proximal end portion 612, and a distal end portion 614. The central body portion 610 is sized for extending through natural lumens of the body to the implantation site. For example, the central body portion 610 may be sized and shaped for passing through the urethra to the bladder. The distal end portion 614 is configured for traversing the natural lumens to reach the implantation site without causing damage or trauma to the body. For example, the distal end portion 614 may traverse the urethra without damaging its walls. The proximal end portion 612 remains outside of the body, providing access for a user, such as a medical professional, or an interface for equipment, such as medical equipment.

The dimensions of the deployment instrument 600 are selected based at least in part on the dimensions of the anatomy that the deployment instrument 600 is designed to navigate. In embodiments in which the deployment instrument 600 extends through the urethra to the bladder, for example, the central body portion 610 may have a length in the range of about 30 cm to about 45 cm and an outer diameter in the range of about 3 mm to about 7 mm. An inner diameter of the central body portion 610 is selected based at least in part on the dimensions of the device that the deployment instrument 600 is designed to implant. For example, the deployment instrument 600 can be used to implant an embodiment of the drug delivery device 100 shown in FIG. 1, in which case the inner diameter of the central body portion 610 may be in the range of about 2 mm to about 5 mm. Thus, the wall 602 of the central body portion 610 is relatively thin. However, the wall 602 is formed from a material that has a column strength suited to prevent buckling despite its thin nature, yet has a flexibility suited for navigating tortuous pathways through natural body lumens such as the urethra. The material also is biocompatible and with materials used to form the device and/or the stylet. The material is also amenable to suitable sterilization processes.

FIG. 7 illustrates the proximal end portion 612 of the deployment instrument 600 in greater detail. The proximal end portion 612 is suited for interfacing with a user or equipment. In some embodiments, the proximal end portion 612 includes a handle 616 that can be grasped by a user. The handle can be integrally formed with the deployment instrument 600, or the handle can be attached separately, such as by bonding. The proximal end portion 612 also can be configured for receiving the device, a stylet, or a flow of liquid, as described below. For example, the proximal end portion 612 can have an entry opening 618 that permits inserting the device, stylet, or a fluid source into the deployment instrument 600. The entry opening 618 may be relatively wider than the interior passageway 604, and extending away from the entry opening 618, the interior passageway 604 may taper. The wider entry opening 618 facilitates easy insertion of the device, the stylet, or the fluid source.

In other embodiments, the proximal end portion 612 is associated with a luer connector. The luer connector facilitates forming a relatively water-tight connection between the deployment instrument 600 and an external fluid source, such as a syringe. Such a configuration is useful in cases in which fluid is introduced into the deployment instrument 600 to drive the device toward the implantation site, as described below.

FIG. 8 illustrates the distal end portion 614 of the deployment instrument 600 in greater detail. The distal end portion 614 includes at least one exit opening 620 that permits releasing the device into the implantation site, such as the bladder. The exit opening 620 is in communication with the interior passageway 604 through the deployment instrument 600 in a manner that facilitates smooth passage of the device from the interior passageway 604 through the exit opening 620. Because the distal end portion 614 leads during insertion of the deployment instrument 600, the distal end portion 614 is configured to navigate tortuous pathways through the body. The distal end portion 614 is strong enough to permit driving the deployment instrument 600 through the natural lumens of the body without buckling or jamming, and yet is flexible enough to minimize or avoid damaging the tissue lumen walls. For example, the distal end portion 614 may facilitate driving the deployment instrument 600 through the urethra to the bladder.

In particular, the distal tip portion 614 includes a substantially solid distal end 622. The solid distal end 622 facilitates driving the catheter forward without buckling. So that the distal end 622 is solid, the interior passageway 604 stops short of the distal end 622. More particularly, the interior passageway 604 curves toward a side of the deployment instrument and terminates at the exit opening 620, which is formed on the side of the distal tip portion 614 through the wall 602. The curvature of the interior passageway 604 guides the device out of the deployment instrument 600 through the exit opening 620. The distal end 622 also has a rounded exterior surface that facilitates driving the deployment instrument forward without causing trauma to the surrounding anatomy, such as the walls of the urethra. Below the rounded exterior surface, the solid distal end 622 may be integrally formed with the deployment instrument 600 from a single material or may be reinforced or filled with one or more reinforcing materials. A reinforced material also may be positioned about the rounded exterior surface of the distal end 622. In the illustrated embodiment, the exit opening 620 is positioned rearward of the rounded exterior surface, although the exit opening 620 may be at least partially positioned on the rounded exterior surface in some embodiments.

The distal tip portion 614 can be formed in a variety of manners. For example, the distal tip portion 614 can be molded or extruded. In one embodiment, the distal tip portion is overmolded onto the catheter. In some embodiments, the distal tip portion 614 can be an integral portion of the catheter, while in other embodiments, the distal tip portion 614 can be a separate, reinforced tip that is attached to the central body portion 610 of the deployment instrument 600.

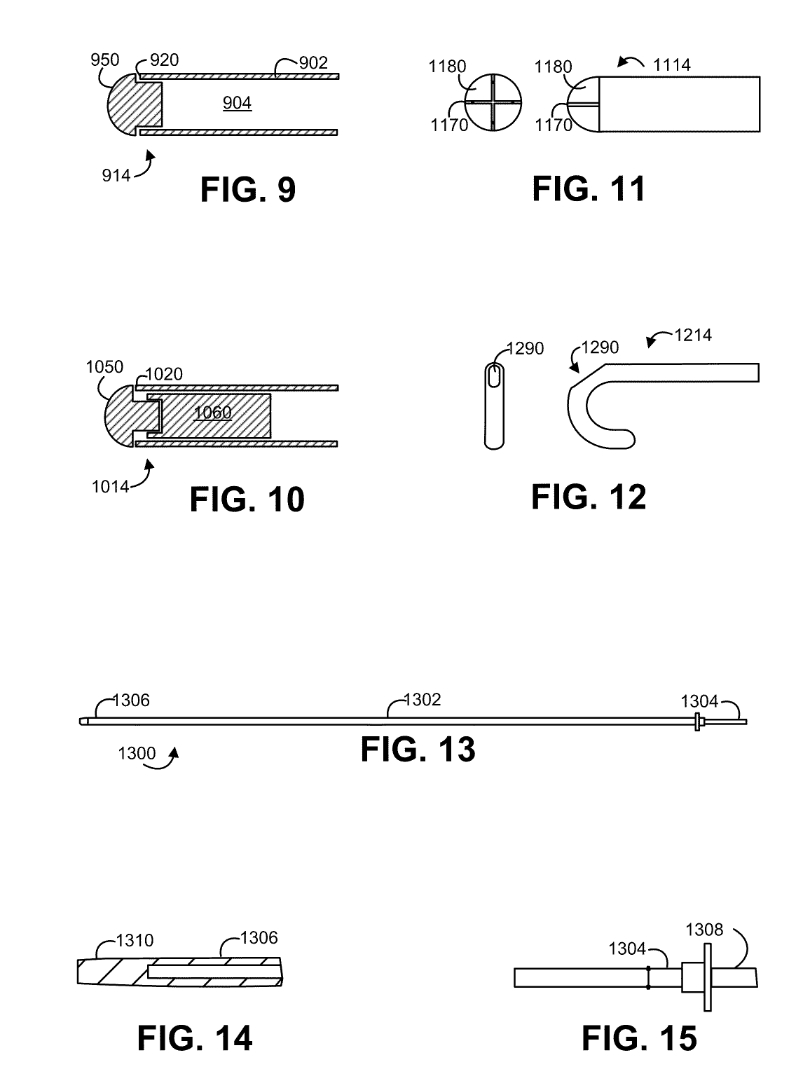

The distal tip portion 614 of the deployment instrument 600 can have other configurations, embodiments of which are shown in FIGS. 9-12. FIG. 9 is a cross-sectional plan view of another embodiment of a distal tip portion 914 that is suited for driving the deployment instrument forward through the urethra, yet permits passing the implantable device into the bladder. As shown, a cap member 950 is removably attached to the distal tip portion 914. The cap member 950 is inserted into the interior passageway 904 to close the exit opening 920, which is formed through a distal end of the wall 902. The cap member 950 includes a distal exterior side that is exposed on an outside of the deployment instrument, and a proximal interior side that is positioned in the interior passageway 904. The cap member 950 may be rounded on its distal exterior side. The rounded exterior surface facilitates driving the deployment instrument forward without causing trauma. Between the exterior surface and the interior surface the cap member 950 has a substantially solid body. The body impedes buckling of the deployment instrument as it is driven through the urethra. On its proximal interior side, the cap member 950 is sized and shaped to mate with and close the exit opening 920 and an adjacent portion of the interior passageway 904. The cap member 950 is removably associated with the exit opening 920 so that the cap member 950 can be removed during the implantation procedure, permitting the device to pass through the exit opening 920. For example, as the device is pushed through the interior passageway 904 toward the exit opening 920, the device may forcibly release the cap member 950 from the deployment instrument. In some embodiments, the cap member 950 is formed from a bioerodible or resorbable material, so that the cap member 950 naturally degrades after release into the body. Examples of materials that are suitably strong yet resorbable include poly(glycerol sebacate) (PGS), poly(lactide-co-glycolide) (PLGA), poly(glycolic acid) (PGA), poly(lactic acid) (PLA) and collagen. In other embodiments, the cap member 950 may be tethered to the deployment instrument, so that the cap member 950 is removed from the body upon removal of the deployment instrument.

FIG. 10 is a cross-sectional plan view of yet another embodiment of a deployment instrument distal tip portion 1014. Like the embodiment shown in FIG. 9, the distal tip portion 1014 includes a cap member 1050. The cap member 1050 may be substantially solid and has a rounded exterior surface on its distal exterior side, which facilitates insertion without buckling or trauma. On its proximal interior side, the cap member 1050 is sized and shaped to mate with and close the exit opening 1020, the cap member 1050 being removably associated with the exit opening 1020 for removal during the implantation procedure. A proximal end of the cap member 1050 is seated in and engages a distal end of the implantable device 1060. Before the deployment instrument is inserted into the body, the implantable device 1060 is loaded into the deployment instrument at the distal end, and the cap member 1050 is attached to the device 1060 to close the exit opening 1020. During the implantation procedure, the cap member 1050 becomes removed from the exit opening 1020 as the implantable device 1060 is pushed forward through the deployment instrument. The cap member 1050 may be formed from a bioerodible or resorbable material, as described above, so that the cap member 1050 does not need to be removed from the body. Alternatively, the cap member 1050 may remain attached to the implantable device 1060 and may be removed with the implantable device following completion of treatment.

FIG. 11 is a side view of another embodiment of a deployment instrument distal tip portion 1114. As shown, the wall of the deployment instrument is rounded about the distal end. Slits 1170 are formed through the rounded portion to define a series of leaves 1180 in the wall about the distal end. The leaves 1180 are generally in a closed position, as shown in FIG. 11, but the slits 1170 permit the leaves 1180 to open. As the implantable device is pushed forward through the distal tip portion 1114 during the implantation procedure, the leaves 1180 open outward so that the implantable device can exit the deployment instrument. Once the implantable device has exited the deployment instrument, the leaves 1180 return to the closed position.

FIG. 12 is a side view of an embodiment of a deployment instrument distal tip 1214 having a coude tip. The shape of the coude tip may be suited for navigating the male anatomy, particularly near the prostate. The coude tip may have an exit opening 1290 along a forward face, so that the device can exit the catheter.

In some embodiments, the deployment system further includes a stylet that can be used to drive the implantable device through at least a portion of the deployment instrument and into the implantation site. FIG. 13 is a plan view of an embodiment of a stylet 1300. Like the deployment instrument, the stylet 1300 includes a central body portion 1302, a proximal end portion 1304, and a distal end portion 1306. The central body portion 1302 is configured to extend through the interior passageway of the deployment instrument to reach the implantation site. The proximal end portion 1304 remains outside of the body and is configured for receiving a driving force from a user or apparatus. The distal end portion 1306 is configured for driving the device from the exit opening of the deployment instrument.

In some embodiments, the implantable device is loaded into the deployment instrument through the exit opening on its distal end portion. The implantable device travels through the urethra as the deployment instrument is inserted into the body, and subsequently, the stylet 1300 is used to drive the implantable device through the exit opening into the body. In other embodiments, the implantable device is loaded into the deployment instrument through the entry opening on its proximal end portion, either before or after the deployment instrument is inserted into the body, and subsequently the stylet 1300 is used to drive the implantable device through the deployment instrument and from the exit opening into the body. In still other embodiments, the implantable device is loaded into the deployment instrument through a slit along its central body portion. The implantable device travels partially through the urethra as the deployment instrument is inserted into the body, and subsequently, the stylet 1300 is used to drive the implantable device through the remaining portion of the deployment instrument and from the exit opening into the body.

The overall shape and configuration of the stylet 1300 permits applying the driving force to the implantable device without substantial friction between the deployment instrument and the stylet 1300 and without the stylet 1300 causing trauma to the deployment instrument or the implantable device.

The dimensions of the stylet 1300 are selected based at least in part on the dimensions of the deployment instrument and the anatomy that the deployment instrument is designed to navigate. Particularly, the stylet 1300 may be slightly longer than the deployment instrument, so that the stylet 1300 can extend fully through the deployment instrument. Once inserted into the deployment instrument, the stylet proximal end portion 1304 is exposed outside of the proximal end portion of the deployment instrument, the stylet central body portion 1302 extends through the central body portion of the deployment instrument, and stylet distal end portion 1306 reaches to the distal end portion of the deployment instrument. Such a configuration permits inserting the stylet 1300 along the full length of the deployment instrument to drive the implantable device from the exit opening on the distal end of the deployment instrument. An outer diameter of the stylet 1300 is smaller than an inner diameter of the deployment instrument, so that the stylet 1300 can be inserted into the deployment instrument without having to overcome the force of friction along the length of the entire deployment instrument. In embodiments in which the deployment instrument has the dimensions described above, for extending through the urethra to the bladder, for example, the central body portion 1302 of the stylet 1300 may have a length in the range of about 35 cm to about 50 cm and an outer diameter in the range of about 2 mm to about 5 mm.

The stylet 1300 is formed from a material that has a column strength suited to reduce buckling, yet has a flexibility suited for navigating through the deployment instrument. The material is biocompatible and is compatible with the materials that are used to form the device and/or the catheter. The material is also amenable to sterilization procedures, such as gamma irradiation or ethylene oxide sterilization. Examples of suitable materials include both metals and polymers, such as stainless steel, cobalt-chromium-based alloys, titanium based alloys, Nitinol, polyethylene (PE), polytetrafluoroethylene (PTFE), styrenic thermoplastic elastomer (STE), silicone, latex rubber, polyurethane, Silitek®, C-flex®, Percuflex®, Tecoflex®, or combinations thereof. The stylet 1300 can be formed by extrusion, molding or other suitable manufacturing processes.

In the illustrated embodiment, the stylet 1300 has a substantially cylindrical outer surface, although other configurations are possible. The proximal end portion 1304 of the stylet 1300 is suited for interfacing with a user or equipment. In some embodiments, the proximal end portion 1304 is a handle 1308 that can be grasped by a user. The handle 1308 can be integrally formed with the stylet 1300, or the handle 1308 can be attached separately, such as by bonding. An example of a handle 1308 is shown in FIG. 15, although other configurations are possible.

For flexibility, the stylet 1300 may have a relatively hollow interior along at least a portion of its length, while the distal end portion 1306 may be substantially solid, or reinforced, to facilitate transferring the driving force to the device. An example is shown in FIG. 14, which illustrates the stylet 1300 having a hollow interior that terminates along the distal end portion 1306 so that the stylet 1300 has a substantially solid distal tip 1310.

The distal end portion 1306 of the stylet 1300 can have a range of configurations. In some embodiments, the distal end portion 1306 has an increased cross-sectional area in comparison to the central body portion 1302. Particularly, the central body portion 1302 has a reduced cross-sectional area to facilitate navigating turns through the deployment instrument with reduced friction, while the distal end portion 1306 has an increased cross-sectional area to facilitate transferring the driving force to the implantable device. The increased cross-sectional area increases the contact surface between the distal end portion 1306 and the implantable device. Further, the increased cross-sectional area of the distal end portion 1306 reduces the likelihood of the stylet 1300 traveling past the implantable device and potentially pinching or trapping the implantable device against the wall of the deployment instrument.

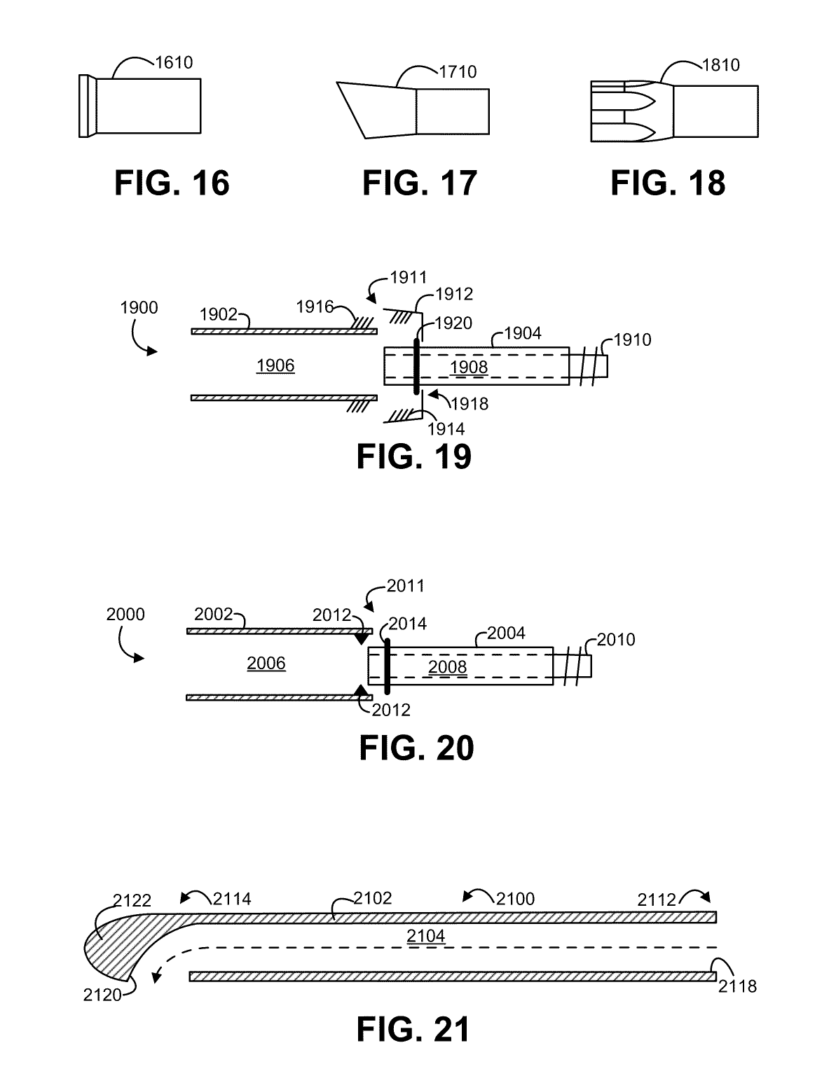

Examples of distal end portions for the stylet 1300 are shown in FIGS. 16-18. FIG. 16 is a side view of a cupped-shaped distal tip portion 1610, which has a cupped-shaped surface at its distal end. The cupped-shaped surface has a larger cross-sectional area than the central body portion of the stylet. Thus, the cupped-shaped surface is suited for contacting the implantable device and transferring the driving force to the implantable device. In embodiments, the cupped-shaped surface has a cross-sectional area that is about the same as, but is slightly smaller than, the inner diameter of the deployment instrument, which reduces the likelihood of the stylet traveling past the implantable device without engaging the implantable device, and yet does not create unsuitable friction between the cupped-shaped surface and the wall of the deployment instrument. Although the surface is shown and described as being cupped-shaped, the surface can be convex, concave, or flat depending on the embodiment. For example, FIG. 17 is side view of an angled distal tip portion 1710. The angled distal tip portion 1710 has a increased cross-sectional area in comparison to the stylet central body portion, but unlike the cupped-shaped distal tip portion 1610, the distal surface of the angled distal tip portion 1710 is flat and angled.

FIG. 18 is side view of a fluted distal tip portion 1810. The fluted distal tip portion 1810 is similar to the cupped-shaped distal tip portion 1610, in that the fluted distal tip portion 1810 has an increased cross-sectional area at a distal end for contacting the implantable device and driving the device forward. The fluted distal tip portion 1810 also has a fluted exterior, which permits fluid to drain past.

In some embodiments, the deployment instrument is designed to receive a stream of fluid that drives the implantable device into the implantation site. In such embodiments, the deployment instrument may connect to a fluid source, such as syringe, so that the stream of fluid can be driven into the deployment instrument. The deployment instrument may have a luer connector that facilitates operably associating the deployment instrument with the fluid source, although other configurations are possible.

FIG. 19 is a cross-sectional plan view of an embodiment of a deployment system 1900 that includes a deployment catheter 1902 and a stylet 1904. The deployment catheter 1902 and stylet 1904 are movable with reference to each other, so that the stylet 1904 can be used to push the implantable device toward the bladder. The deployment catheter 1902 and stylet 1904 also form a continuous fluid path for delivering fluid to and from the bladder, such as for distending or draining the bladder, yet the deployment catheter 1902 and stylet 1904 can be sealed with reference to each other to reduce leakage.

In particular, the stylet 1904 has a reduced cross-section with reference to the deployment catheter 1902 so that the stylet 1904 can fit inside an internal lumen 1906 of the deployment catheter 1902. The stylet 1904 also includes an internal lumen or channel 1908, and when the stylet 1904 is inserted into the deployment catheter 1902, the internal lumen 1908 of the stylet 1904 is in fluid communication with the internal lumen 1906 of the deployment instrument 1902. A fluid port 1910, such as a luer connector, slip tip, or other suitable fluid source connection means, may be formed on a proximal end of the stylet 1904 for introducing fluid to, or receiving fluid from, the stylet internal lumen 1908. A seal 1911 is formed between the deployment catheter 1902 and stylet 1904, reducing leakage. In the illustrated embodiment, for example, the seal includes a threaded cap 1912 positioned on the distal end of the stylet 1904. The threaded cap 1912 includes internal threads 1914 that are configured to engage external threads 1916 about the proximal end of the deployment catheter 1902. The threaded cap 1912 has an opening 1918 that permits the stylet 1904 to pass through the threaded cap 1912 into the deployment catheter 1902. An O-ring 1920 is positioned adjacent to the opening 1918 to reduce leakage.

FIG. 20 is a cross-sectional plan view of another embodiment of a deployment system 2000 that includes a deployment catheter 2002 and a stylet 2004. Like the deployment system 1900, the deployment catheter 2002 and stylet 2004 can move with reference to each other, form a continuous fluid path for delivering fluid to and from the bladder, and can be sealed with reference to each other to reduce leakage. The stylet 2004 has a reduced cross-section with reference to the deployment catheter 2002 so that the stylet 2004 can fit inside an internal lumen 2006 of the deployment catheter 2002. The stylet 2004 also includes an internal lumen 2008 that is in fluid communication with the internal lumen 2006 of the deployment instrument 2002 when the stylet 2004 is inserted into the deployment catheter 2002. The stylet 2004 also includes the fluid port 2010, such as a luer connector, slip tip, or other suitable fluid source connection means, for introducing fluid to or receiving fluid from, the stylet internal lumen 2008, and a seal 2011 is formed between the deployment catheter 2002 and the stylet 2004 to reduce leakage. In FIG. 20, the seal 2011 includes a protruding rim 2012 or shoulder concentrically positioned about the interior of the deployment catheter 2002 and an O-ring or seal 2014 concentrically positioned about the exterior of the stylet 2004. The rim 2012 and O-ring 2014 are sized to engage each other as the stylet 2004 is driven through the deployment catheter 2002, forming a seal 2011 that reduces leakage.

FIG. 21 is a cross-sectional plan view of such an embodiment of a deployment instrument 2100. The deployment instrument 2100 generally includes a catheter having an internal lumen 2104, a fluid introduction port 2118 located at its proximal end, and an exit opening 2120 located at a distal end. Both the fluid introduction port 2118 and the exit opening 2120 are in fluid communication with the internal lumen 2104. In the illustrated embodiment, the deployment instrument 2100 has a reinforced distal end 2122, much like the deployment instrument shown in FIG. 6. The distal opening 2120 is positioned on the distal end of the deployment instrument 2100 offset from its longitudinal axis. The offset placement of the distal opening 2120 permits configuring the distal tip 2122 for advancing through the urethra without causing trauma. For example, the distal tip 2122 may include a rounded portion, which may be solid and/or reinforced. The distal opening 2120 may be positioned on the rounded portion offset from center or the distal opening 2120 may be formed along a side of the deployment instrument through the wall 2102 near the rounded portion. Other configurations are possible.

In use, the deployment instrument 2100 is inserted through the urethra. The implantable device may be pre-loaded into the deployment instrument 2100, positioned near the distal end portion 2114, so that introducing the deployment instrument 2100 into the body simultaneously introduces the implantable device into the body. However, other configurations are possible. For example, the implantable device may be loaded into the deployment instrument 2100 after the deployment instrument 2100 is introduced into the body, and the implantable device may be advanced along the length of the deployment instrument 2100 to the distal end, using a stream of fluid, a push rod or stylet, or a combination thereof.

Once the distal end portion 2114 of the deployment instrument 2100 is positioned in the bladder, urine may enter the distal opening 2120 and may travel along the length of the internal lumen 2104 to the fluid introduction port 2118. The return of urine through the fluid introduction port 2118 indicates proper placement of the deployment instrument 2100 in the body with the distal end portion 2114 located in the bladder.

A volume of water or other suitable fluid then may be introduced into the bladder to form a cushion for the implantable device. For example, a syringe of water may be operably associated with the fluid introduction port 2118. The syringe may block the fluid introduction port 2118 to prevent the further escape of urine. The syringe may be depressed to introduce water into the bladder, inflating the bladder to provide a cushion for the implantable device. About 50 to 60 mL of water may be introduced into the bladder, although other volumes of water may be used. In cases in which the implantable device is positioned in the internal lumen 2104, the water may travel about spaces between the implantable device and the wall of the deployment instrument 2100. The force of the water traveling through the internal lumen 2104 may or may not advance the implantable device forward.

Once the bladder has been inflated, the proximal end portion 2112 of the deployment instrument may be blocked adjacent to the fluid introduction port 2118 to prevent the return of water or urine as the syringe of water is removed. For example, an external clamp may be applied to the deployment catheter. One suitable clamp is an Easy Reopening Closure Clamp made by the Qosina Corporation of Edgewood, N.Y. The clamp may not cause permanent deformation to the deployment instrument 2100 due to the compliant nature of its walls 2102.

A syringe of fluid is then placed in fluid communication with the fluid introduction port 2118. The clamp is removed and the syringe is pushed to eject the fluid. The fluid is communicated through the fluid introduction port 2118 and along the length of the deployment instrument 2100 to drive the implantable device from the distal end of the deployment instrument 2100 into the bladder. Thereby, the implantable device is implanted. Thereafter, a push-rod or stylet may be inserted through the deployment instrument 2100 to verify that the implantable device has exited the deployment instrument 2100 into the bladder.

The amount of fluid needed to implant the implantable device may vary depending on the dimensions of the deployment instrument 2100. For example, about 10 to 20 mL of fluid may be injected. Suitable fluids are relatively viscous, water-soluble, biocompatible, incompressible, and do not negatively interact with the deployment instrument 2100 or the implantable device. Suitable fluids also may not interfere with any drug delivered from the implantable device once implanted. In one example, the fluid may comprise a lubricant, such as Surgilube brand lubricant, which is manufactured by Fougera Inc. of Melville, N.Y. Other suitably viscous fluids also may be used. For example, the fluid may have a viscosity that exceeds the viscosity of water, at ambient or approximately body temperature. Injecting a suitably viscous fluid into the deployment instrument 2100 may drive the implantable device forward despite the weight of the implantable device and the force of friction, such as friction between the implantable device and the wall 2102 of the deployment instrument 2100. However, less viscous fluids, such as water or biocompatible aqueous solutions, may be used. Although the deployment instrument is described as receiving a stream of fluid from a syringe, other fluid sources can be used.

The method of implanting a drug delivery device described above with reference to FIG. 21 is merely one example, and other implantation methods are possible. For example, it may not be necessary to introduce water into the bladder before the implantable device is implanted. It also may not be necessary to verify implantation of the drug delivery device with a push-rod or stylet. Further, the deployment instrument 2100 may have other configurations. For example, the deployment catheter 2100 may have more than one port on its proximal end. Also, the port may be associated with a valve that can be moved from an open position to a closed position to prevent fluid from exiting the port. In such embodiments, it may not be necessary to clamp the deployment instrument during the implantation procedure. The deployment instrument also may have a luer connector or a slip tip on one or more of the ports to facilitate operably associating the deployment instrument with a fluid source. In some embodiments, the deployment instrument is divided into a number of discrete lumens, such as two or three lumens. Each discrete lumen may be associated with one or more ports. Further, each port may be operably associated with valve. A range of combinations of lumens, ports, and valves can be used to achieve a suitable deployment instrument.

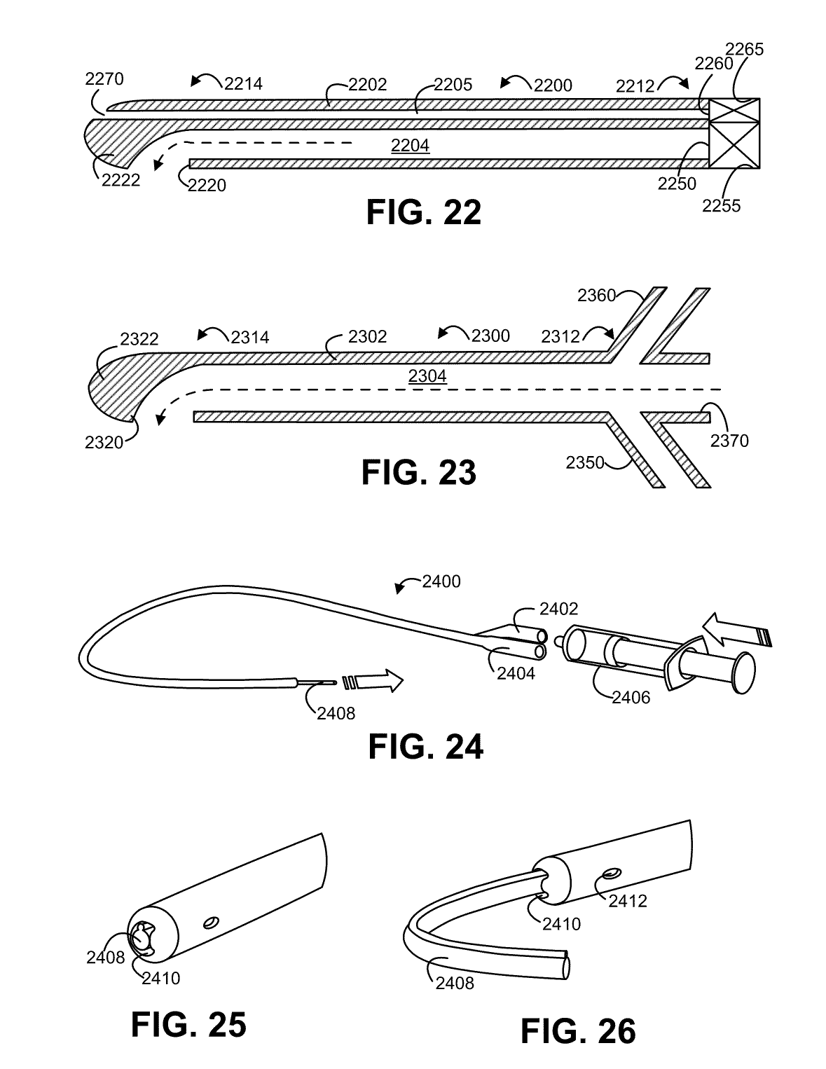

For example, FIG. 22 illustrates a deployment instrument 2200 having a main lumen 2204 in communication with a main port 2250 and a urine return lumen 2205 in communication with a urine return port 2260. Each port 2250, 2260 may be associated with a valve 2255, 2265 that can be opened or closed to permit or prevent the flow of fluid there through. For example, the valve 2255 on the main port 2250 can be opened to permit the introduction of fluid into the main lumen 2204 for the purpose of driving the implantable device forward, and the valve 2255 can be closed to prevent fluid from returning through the main port 2250. The valve 2265 on the urine return port 2260 may be opened to permit the return of urine through the urine return lumen 2205 and can be closed to prevent the continued return of urine once placement of the deployment instrument 2200 in the bladder has been verified.

In use, the deployment instrument 2200 may be inserted through the urethra with the valve 2255 on the main port 2250 closed and the valve 2265 on the urine return port 2260 open. Thus, when the distal end portion 2214 of the deployment instrument 2200 becomes positioned in the bladder, urine can enter the urine return opening 2270 on the distal top of the deployment instrument 2200. The urine travels through the urine return lumen 2205 and from the urine return port 2260 to indicate placement of the distal tip of the deployment instrument 2200 in the bladder. Once placement is confirmed, the valve 2265 associated with the urine return port 2260 may be closed to prevent the continued return of urine. A syringe or other fluid introduction mechanism is then operably associated with the main port 2250. The valve 2255 on the main port 2250 is opened, and fluid is introduced from the syringe through the main port 2250 to drive the implantable device into the bladder. The main port 2250 is then closed. In some embodiments, delivery of the implantable device from the deployment instrument 2200 may be verified using a push-rod or stylet. The push rod is inserted into the main port 2250, the valve 2255 is opened, and the push rod is inserted along the length of the deployment instrument 2200 to verify delivery of the implantable device from the deployment instrument 2200. It should be noted that the valves 2255, 2265 may be augmented or substituted with external clamps in these and in other embodiments. The external clamp may be removably positioned about the ports 2250, 2260 to permit or prevent the flow of fluid as needed. In these and in other embodiments, the ports 2250, 2260 may be angled or spaced apart from each other, as shown in FIG. 23.

FIG. 23 illustrates a deployment instrument 2300 having a main lumen 2304 operably associated with a fluid introduction port 2350, a urine return port 2360, and a push-rod port 2370. The use of the deployment instrument 2300 is similar to the use of the deployment instrument 2200 shown in FIG. 22. Each port may be associated with a valve that can be opened or closed to permit or prevent the flow of fluid through the ports. For example, the valve on the fluid introduction port 2350 can be opened to permit the introduction of fluid into the main lumen 2304 for the purpose of driving the implantable device forward and can be closed to prevent the introduced fluid from returning through the fluid introduction port 2350. The valve on the urine return port 2360 also may be opened to permit the return of urine, indicating placement of the deployment instrument 2300 in the bladder, and can be closed to prevent the continued return of urine once placement has been verified. The valve on the push-rod port 2370 may be opened to introduce a push-rod into the main lumen 2304 for the purpose of verifying the implantable device has exited the deployment instrument 2300 or for driving the implantable device from the deployment instrument 2300, and may be closed thereafter.

In use, the urine return port 2360 may be opened and the fluid introduction and push-rod ports 2350, 2370 may be closed as the deployment instrument 2300 is deployed through the urethra, so that urine can return through the main lumen 2304 and from the urine return port 2360 to indicate placement of the distal tip portion 2314 in the bladder. Once placement is confirmed, the valve associated with the urine return port 2360 may be closed. A syringe or other fluid introduction mechanism is associated with the fluid introduction port 2350. The valve on the fluid introduction port 2350 is opened, and fluid is introduced from the syringe through the fluid introduction port 2350 to drive the implantable device into the bladder. The fluid introduction port 2350 is then closed, and a push rod is inserted into the push-rod port 2370. The valve on the push-rod port 2370 is opened, and the push rod is inserted along the length of the deployment instrument 2300 to verify delivery of the implantable device from the deployment instrument 2300. However, verification with the push rod is not necessary, in which case the push-rod port 2370 may be omitted. It should be noted that the valves may be substituted with external clamps in these and in other embodiments. The external clamp may be removably positioned about the deployment instrument 2300 to permit or prevent the flow of fluid as needed. Additionally, some or all of the ports may share a single valve positioned upstream from the ports on the deployment instrument 2300.

FIG. 24 illustrates another embodiment of a deployment instrument 2400 for delivering an implantable device to the bladder. The deployment instrument 2400 generally includes a catheter having a fluid introduction port 2402 located at its proximal end, and a syringe of fluid 2406 in fluid communication with the fluid introduction port 2402. In use, the implantable device 2408 is pre-loaded into an exit opening 2410 on the distal end of the deployment instrument 2400, as shown in FIG. 25. The exit opening 2410 also may be formed through a side of the deployment instrument 2400 so that the distal end can be reinforced, as described above. The deployment instrument 2400 is then introduced into the body. When the syringe 2406 is pushed to eject the fluid, the fluid is communicated through the fluid introduction port 2402 and along the length of the deployment instrument to drive the implantable device 2408 from the exit opening 2410 in the distal end of the deployment instrument into the bladder, as shown in FIGS. 24 and 26. Thereby, the implantable device 2408 is implanted.

In some embodiments, the deployment instrument 2400 is divided into two discrete lumens. A main lumen is sized for receiving the implantable device 2408, which is loaded into the main lumen. In some embodiments, the cross-sectional shape of the main lumen may match the cross-sectional shape of the implantable device. The main lumen is also in fluid communication with the fluid introduction port 2402. Thus, fluid introduced through the fluid introduction port 2402 travels through the main lumen to drive the implantable device 2408 into the bladder.

A secondary lumen is also provided. The secondary lumen extends from a secondary opening 2412 at a distal end of the deployment instrument 2400 to a fluid exit port 2404 positioned at the proximal end of the deployment instrument 2400, such as adjacent to the fluid introduction port 2402. The secondary lumen can transport fluid from the distal end of the deployment instrument 2400 to the proximal end and out of the fluid exit port 2404. In use, when the distal end of the deployment instrument 2400 enters the bladder, urine from the bladder enters the secondary opening 2412 at the distal end and travels through the secondary lumen to the fluid exit port 2404 on the proximal end. The return of urine through the secondary lumen indicates the distal end has become positioned in the bladder. In such embodiments, the secondary opening 2412 can be thought of as a urine entry opening, the secondary lumen can be thought of as a urine return lumen, and the fluid exit port 2404 can be thought of as a urine exit port.

In some embodiments, the urine entry opening 2412 can be formed through a distal end surface of the deployment instrument 2400, so that urine can enter the urine return lumen as soon as the distal end surface of the deployment instrument 2400 has entered the bladder. In other embodiments, the urine entry opening 2412 can be formed through a side of the deployment instrument 2400, rearward of the distal surface by a predetermined distance, so that urine can enter the urine return lumen as soon as the distal end of the deployment instrument 2400 has passed the predetermined distance into the bladder. Such an embodiment in shown in FIG. 26. In some embodiments, the urine return lumen is sized to impede urine flow, so that the urine exit port 2404 can be left unattended. In such embodiments, a vacuum can be applied to the urine exit port 2404 to cause urine to travel through the urine return lumen, so that the user can determine whether the distal end of the deployment instrument 2400 is properly positioned in the bladder. In other embodiments, the urine exit port 2404 may be associated with a plug, or a stop, that is operable to block the return of urine once initial placement of the deployment instrument 2400 has been verified. However, the urine return lumen is not necessary and may be omitted.

In some embodiments, the deployment system 2400 is used in conjunction with a stylet. The lubricant or other suitable fluid is introduced through the fluid introduction port 2402 before the stylet is inserted. Thus, when the stylet is driven along the length of the deployment instrument 2400, the stylet forces the fluid forward, which carries the implantable device 2408 forward and out of the exit opening 2410. In such embodiments, the stylet may have an outer diameter that is slightly smaller than an inner diameter of the main lumen, and the fluid may be relatively incompressible and viscous, so that the fluid does not leak about the stylet or the implantable device 2408 but instead is pushed forward along with the device 2408.

In such embodiments, the implantable device 2408 and the fluid can be positioned in the deployment instrument 2400 before the deployment instrument 2400 is inserted into the body. Once the deployment instrument 2400 is positioned in the body, the stylet is inserted along the length of the deployment instrument 2400 to drive the fluid and the implantable device 2408 forward until the device 2408 exits the exit opening 2410 into the bladder. Alternatively, one or both of the implantable device 2408 and the fluid can be inserted into the deployment instrument 2400 after the deployment instrument 2400 is positioned in the body. The fluid may also be omitted, in which case the stylet directly contacts the implantable device 2408 to drive the device into the bladder.

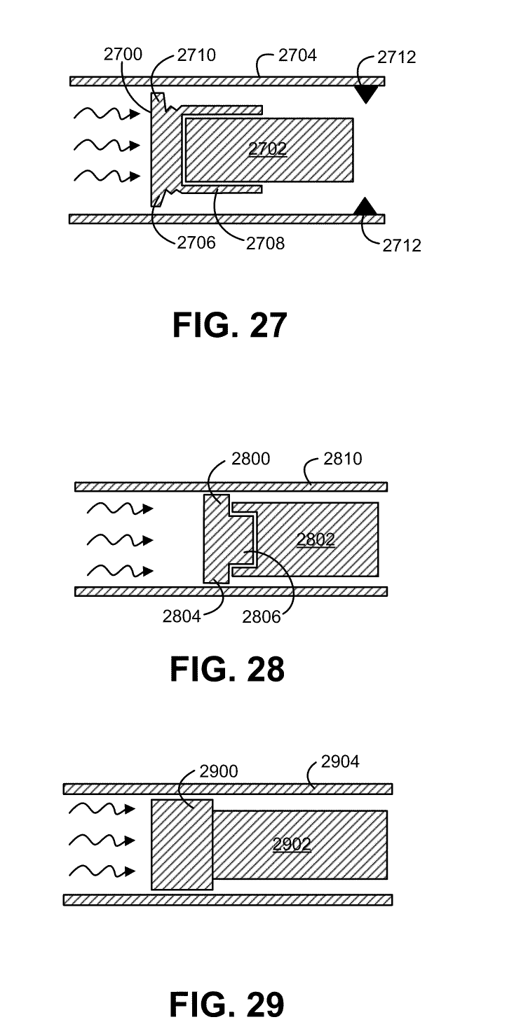

As described above, the implantable device may be implanted by injecting a fluid into a deployment instrument. The fluid may drive the implantable device along the deployment instrument into the bladder. In some embodiments, a movable seal or transfer plug may be positioned between the fluid and implantable device. The movable seal or transfer plug may transfer the driving force of the fluid to the implantable device. In particular, the movable seal or transfer plug may be configured to increase the portion of the available driving force that is transferred directly to the implantable device, such as by reducing the amount of driving fluid that travels past the implantable device without acting on the device to drive it forward. In addition, the movable seal or transfer plug may facilitate using a less viscous driving fluid, such as water. Embodiments of a movable seal or transfer plug are described below with reference to FIGS. 27 through 29.

FIG. 27 is a cross-sectional view of an embodiment of a plunger 2700 that can be positioned about an end of an implantable device 2702 to facilitate driving the implantable device 2702 through the deployment instrument 2704. In particular, the implantable device 2702 may have a smaller cross-section than the deployment instrument 2704. When lubricant is used to drive the implantable device 2702 through the deployment instrument 2704, the lubricant may escape about the implantable device 2702 instead of driving the implantable device 2702 forward. Thus, the plunger 2700 can be used to increase the cross-sectional area of the implantable device 2702 about at least a proximal end so that the lubricant is impeded from passing between the implantable device 2702 and the deployment instrument 2704.

The plunger 2700 is generally formed from a flexible material, such as rubber. The plunger 2700 may be cap-shaped, having a head portion 2706 and a flange portion 2708. The head portion 2706 may have an outer rim 2710 that corresponds in shape and size to the internal lumen of the deployment instrument 2704. The outer rim 2710 contacts the wall of the deployment instrument 2704, forming a seal with the deployment instrument 2704.

The flange portion 2708 defines an opening or cavity that can matingly engage at least a portion of the implantable device 2702. The flange portion 2708 may have an outer cross-section that is smaller than the internal lumen of the deployment instrument 2704 to reduce contact between the plunger 2700 and the wall of the deployment instrument 2704, thereby reducing friction. Also, the flange portion 2708 may not be continuous about its entire circumference to reduce the weight of the plunger 2700 and to further reduce the likelihood of contact with the wall of the deployment instrument 2704.

The deployment instrument 2704 may have at least one stop 2712 configured to impede forward movement of the plunger 2700 past a certain point. The stop 2712 may be a rim or a series of nubs that project from the wall of the deployment instrument 2704 into its lumen.

In use, the plunger 2700 is operably associated with the implantable device 2702 such that the proximal end of the implantable device 2702 becomes seated in the opening in the flange portion 2708. The plunger 2700 and implantable device 2702 are then loaded into the deployment instrument 2704. The outer rim 2710 forms a seal with the wall of the deployment instrument 2704, and fluid is introduced into the deployment instrument 2704. The force of the fluid against the head portion 2706 drives the plunger 2700, and therefore the implantable device 2702, forward. Once the implantable device 2702 has been delivered from the deployment instrument 2704, the outer rim 2710 of the plunger 2700 may contact the stop 2712 to prevent the plunger 2700 from continued forward travel. Thus, the implantable device 2702 may be implanted, but the plunger 2700 may remain in the deployment instrument 2704.

The plunger 2700 impedes the driving fluid from passing between the implantable device 2702 and the wall of the deployment instrument 2704 instead of driving the implantable device 2702 forward. Thus, the implantable device 2702 may be implanted using a lower viscosity fluid, such as water. The driving fluid may not contact the implantable device 2702 during the implantation process due to the presence of the plunger 2700.

FIG. 28 is a cross-sectional view of another embodiment of a plunger 2800 positioned about an end of an implantable device 2802. As shown, the plunger 2800 is generally a cap piece that is attached to the proximal end of the implantable device 2802. The cap has a head portion 2804 and a tip portion 2806. The head portion 2804 corresponds in shape and size to the internal lumen of the deployment instrument 2810, while the tip portion 2806 corresponds in size and shape to an internal lumen in the implantable device 2802. In use, the plunger 2800 is attached to the implantable device 2802 by inserting the tip portion 2806 into the implantable device 2802, and the implantable device 2802 is loaded into the deployment instrument 2810. The head portion 2804 of the plunger 2800 contacts the wall of the deployment instrument 2810, forming a seal, and fluid is introduced into the deployment instrument 2810 to drive the implantable device 2802 forward. The plunger 2800 is implanted with the implantable device 2802 and is either removed or resorbed with the device 2802.

FIG. 29 is side view of another embodiment of a transfer plug or movable seal 2900 that is separate from the implantable device 2902. The transfer plug 2900 has a cross-sectional area that is about the same as the deployment instrument 2904, while the implantable device 2902 has a cross-sectional area that is smaller than the deployment instrument 2904. The transfer plug 2900 may facilitate transferring the driving force of the fluid to the implantable device 2902. The transfer plug 2900 may be attached to the implantable device 2902, or the transfer plug 2900 may be separate from the implantable device 2902, in which case the transfer plug 2900 may be formed from a resorbable material, so that the transfer plug 2900 can be driven into the bladder along with the implantable device 2902 and subsequently is resorbed.

It should be noted that any of the embodiments of plungers, movable seals, or transfer plugs shown and described in FIGS. 27 through 29 can be used in association with a force transfer device, such as a stylet, plunger, or screw, instead of a flow of fluid. In such cases, the plunger, movable seal, or plug may be attached to the force transfer device instead of the implantable device, or the plunger, movable seal, or plug may be separate from both the force transfer device and the implantable device, in which case a resorbable material may be used, although other configurations are possible.

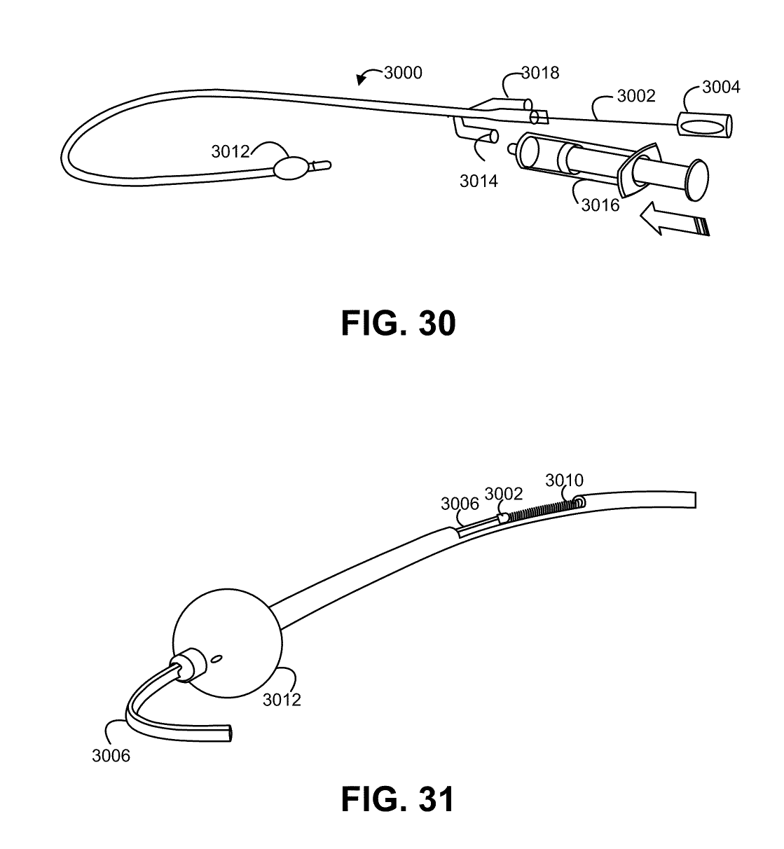

FIG. 30 illustrated another embodiment of a deployment instrument 3000 for delivering an implantable device into the bladder. The deployment instrument 3000 generally includes a plunger 3002 and a handle 3004 operatively connected to the plunger 3002. The handle 3004 is positioned at the proximal end of the deployment instrument 3000. The plunger 3002 extends from the handle 3004 through a main lumen of the deployment instrument 3000. The plunger 3002 is positioned rearward of the distal end of the deployment instrument 3000. In use, the implantable device 3006 is pre-loaded into the distal end of the deployment instrument 3000, and the deployment instrument 3000 is introduced into the body. When the handle 3004 is pushed, the plunger 3002 travels forward along the length of the deployment instrument 3000 to drive the implantable device 3006 from the distal end of the deployment instrument 3000 into the bladder. Thereby, the implantable device 3006 is deployed or implanted.

In certain embodiments, the plunger 3002 is associated with a spring 3010 that is configured to magnify the driving force of the user on the handle 3004. The plunger 3002 and spring 3010 are visible in FIG. 31, which illustrates a perspective partial cut-away view of the distal end portion of the deployment instrument 3000. In some embodiments, the spring 3010 reverses the direction of the driving force. In such embodiments, the implantable device 3006 is driven forward by pulling the handle 3004 rearward.

In some embodiments, the deployment instrument 3000 is a Foley catheter suited for retaining the deployment instrument in the bladder. The deployment instrument 3000 has a balloon 3012 located about its distal end. The balloon 3012 is in fluid communication with a fluid entry port 3014 on the proximal end of the deployment instrument 3000 by way of a secondary lumen through the deployment instrument 3000. In use, fluid introduced through the fluid entry port 3014, such as via a syringe 3016, travels along the secondary lumen to inflate the balloon 3012. Thus, the fluid entry port 3014 can be thought of as a Foley balloon inflation port, and the secondary lumen can be thought of as a Foley balloon inflation lumen. Once the balloon is inflated, the balloon 3012 may impede the deployment instrument 3000 from traveling rearward through the urethra, such that the distal end of the deployment instrument 3000 is retained in the bladder. However, the Foley balloon 3012 and corresponding port 3014 and lumen can be omitted. In the illustrated embodiment, the deployment instrument 3000 further includes a urine return port 3018 in communication with a urine return opening on the distal end of the deployment instrument 3000 via a urine return lumen. The urine return port 3018 facilitates determining when the distal end has become positioned in the bladder so that the Foley balloon 3012 is not inflated prematurely. Thus, the illustrated deployment instrument 3000 has three internal lumens, each of which is in communication with separate openings on distal and proximal ends of the deployment instrument 3000. However, one or both of the Foley balloon inflation lumen and/or the urine return lumen may be omitted, along with its corresponding ports and openings.

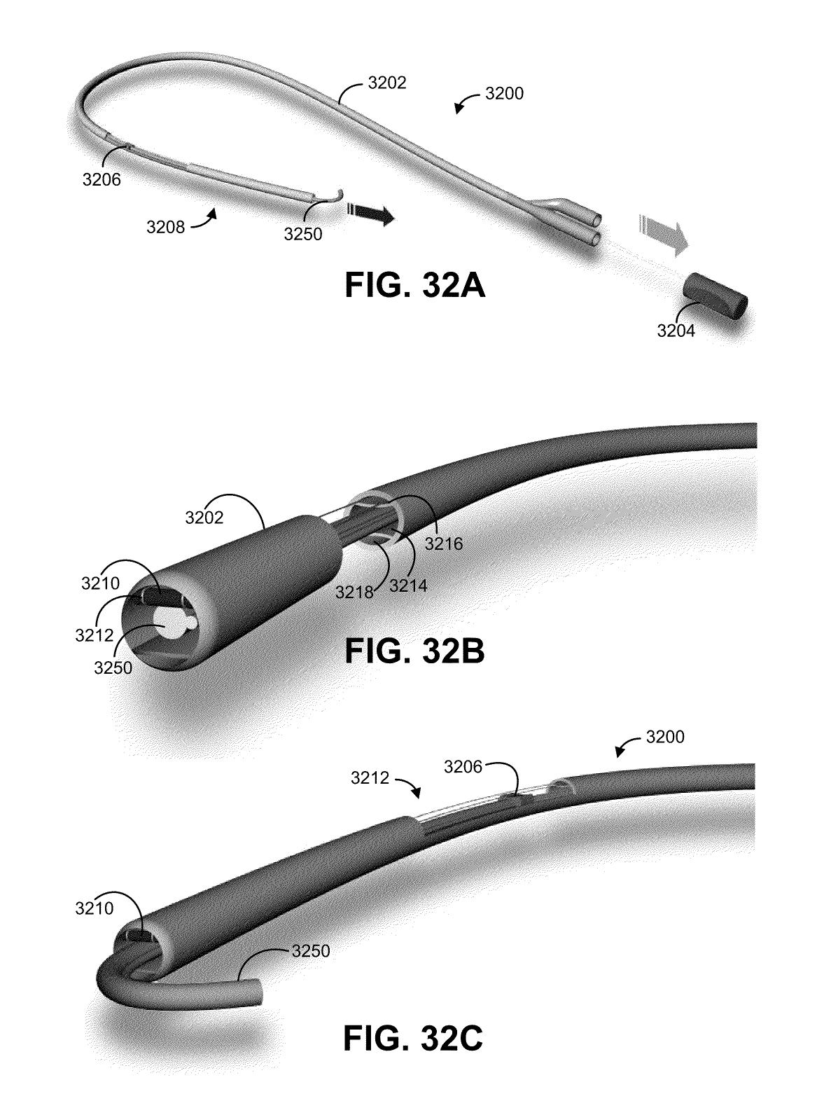

FIG. 32 illustrates an additional embodiment of a deployment device 3200 for delivering an implantable device in the bladder. The deployment device 3200 generally includes a catheter 3202, a handle 3204, a plunger 3206, and a pulley system 3208. The figures are partial cut-away views so that the plunger 3206 and pulley system 3208 are visible. The handle 3204 is operatively connected to the plunger 3206 via the pulley system 3208, so that rearward movement or other actuation of the handle 3204 causes the plunger 3206 to travel forward. In particular, the handle 3204 is positioned at the proximal end of the catheter 3202. The plunger 3206 is positioned along the length of the catheter 3202 somewhere between its proximal and distal ends, as shown in FIG. 32C. The pulley system 3208 includes a pulley roller 3210 located at a distal end of the catheter 3202, and a number of strings or wires 3212. The strings 3212 extend from the handle 3204 at the proximal end, about the pulley roller 3210 at the distal end, to the plunger 3206 therebetween. Thus, when the handle 3204 is pulled rearward, the strings 3212 travel rearward about the pulley roller 3210, pulling the plunger 3206 forward.

In use, the implantable device 3250 is loaded in the deployment device 3200 from the distal end, such that the plunger 3206 is located rearward of the implantable device 3250. When the handle 3204 is pulled as shown in FIG. 32A, the pulley system 3208 causes the plunger 3206 to push the implantable device 3250 from the distal end of the catheter 3202 as shown in FIG. 32C. Thereby, the implantable device 3250 is implanted.

In some embodiments, the catheter 3202 includes at least two separate lumens as shown in FIG. 32B. A main lumen 3214 houses the plunger 3206 and is sized for receiving the implantable device 3250, while a secondary lumen 3216 houses the portion of the strings 3212 that extend from the handle 3204 to the pulley roller 3210. At the distal end of the catheter, the strings 3212 wrap about the pulley roller 3210 and extend rearward through the main lumen to connect to the plunger 3206. However, the strings 3212 need not be housed in a separate lumen in other embodiments.

The illustrated embodiment also includes a urine return lumen 3218 and an associated distal end opening and proximal end port, although the urine return lumen could be omitted. A Foley balloon may also be provided in some embodiments, along with the associated distal and proximal end openings and longitudinal lumen, either alone or in combination with the urine return lumen.

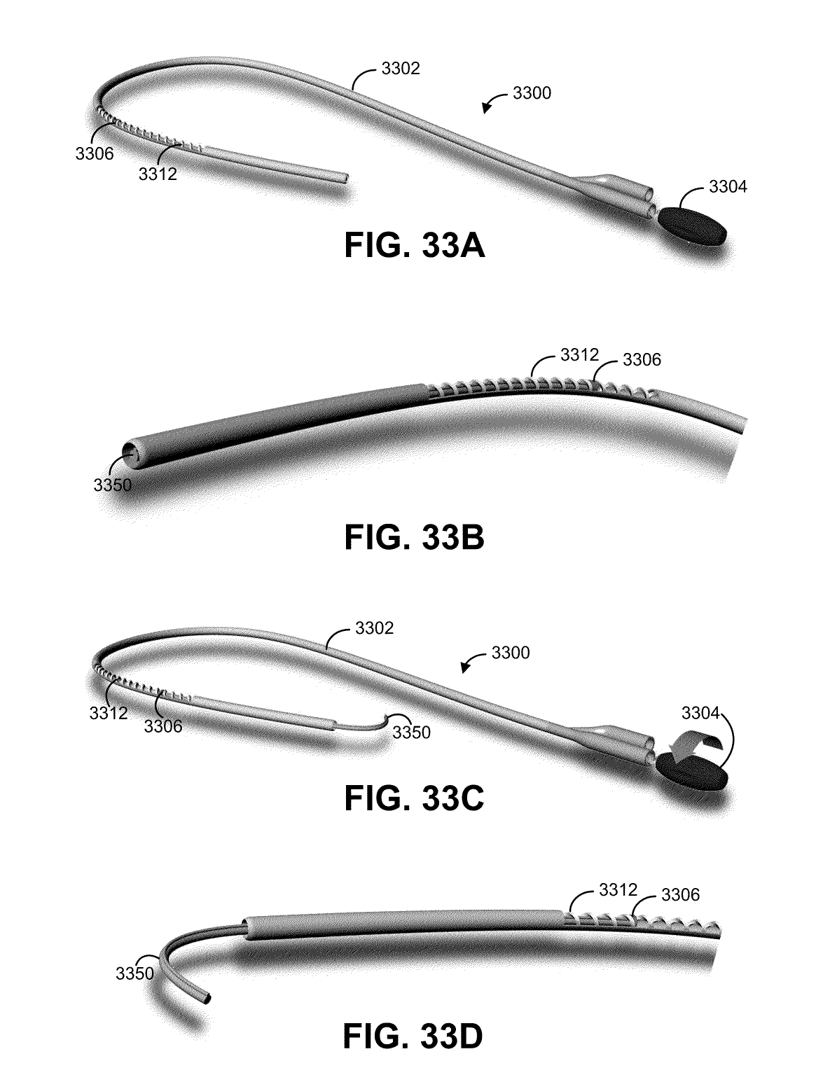

FIG. 33 illustrates another embodiment of a deployment system 3300 for delivering an implantable device into the bladder. Like the embodiment of FIG. 32, the deployment system generally includes a deployment catheter 3302 having a plunger 3306 and a handle 3304 operatively connected to the plunger 3306. The handle 3304 is positioned at the proximal end of the catheter 3302, and the plunger 3306 is positioned rearward of the distal end of the catheter 3302. Unlike the embodiment of FIG. 32, however, the handle 3304 is operatively connected to the plunger 3306 by way of a screw 3312 that extends along at least a portion of the length of the catheter 3302. The plunger 3306 and screw 3312 are visible in FIGS. 33B and 33D, which are partial cut-away, perspective views of the deployment instrument 3300.

In use, the implantable device 3350 is pre-loaded into the distal end of the catheter 3302, and the catheter 3302 is introduced into the body. When the handle 3304 is rotated or is otherwise actuated as shown in FIG. 33C, the screw 3312 turns, causing the plunger 3306 to travel forward along the length of the catheter 3302 to drive the implantable device 3350 from the distal end of the catheter 3302 as shown in FIG. 33D. Thereby, the implantable device 3350 is implanted, such as into the bladder. It should be noted that one or both of a urine return and a Foley balloon can be provided with the embodiment of FIG. 33 even though these are not shown.

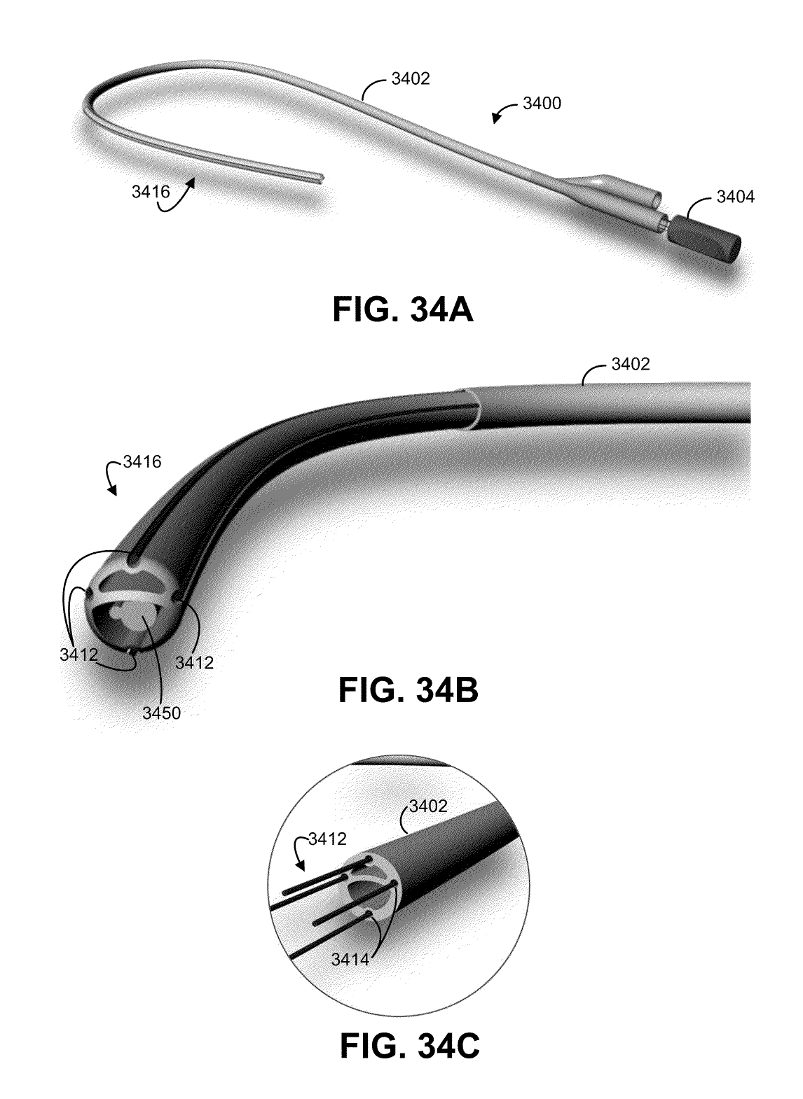

FIG. 34 illustrates another embodiment of a deployment instrument 3400 for delivering an implantable device into the bladder. The deployment instrument 3400 includes a catheter 3402 having a handle 3404 operatively associated with a number of strings 3412. The handle 3404 is located at the proximal end of the catheter 3402, and the strings 3412 extend through channels 3414 in the wall of the catheter 3402 to the distal end. The strings 3412 and channels 3414 can be seen in FIG. 34C, which is a partial, cut-away perspective view of a portion of the catheter 3402. At the distal end, the catheter 3402 is scored or subdivided into a number of leaved end portions 3416 that extend rearward along the length of the catheter 3402 from its distal end. Each of the strings 3412 is fixedly attached to one of the leaved end portions 3416 of the catheter 3402. In use, the implantable device 3450 is pre-loaded into the distal end of the catheter 3402, and the catheter 3402 is introduced into the body. When the handle 3404 is pulled, the strings 3412 are retracted rearward through the channels 3414, pulling the leaved end portions 3416 rearward from the distal end of the catheter 3402. The whole distal end portion of the catheter 3402 splits open, much like a peeled banana, releasing the implantable device 3450 into the bladder. A urine return is illustrated in FIG. 34, although the urine return may be omitted or provided in combination with a Foley balloon in other embodiments.

In some embodiments, the deployment system may include a guide wire. In such embodiments, the implantable device may be configured for deployment over a guide wire. The implantable device may have a bounded opening for receiving the guide wire. The guide wire may be threaded through the urethra until its distal end becomes positioned in the bladder and its proximal end becomes positioned outside of the body, and the implantable device may be threaded over the guide wire. More particularly, the guide wire may pass through the opening in the implantable device, and the implantable device may be pushed along the guide wire into the bladder. The guide wire then may be removed from the urethra, leaving the implantable device implanted in the bladder.

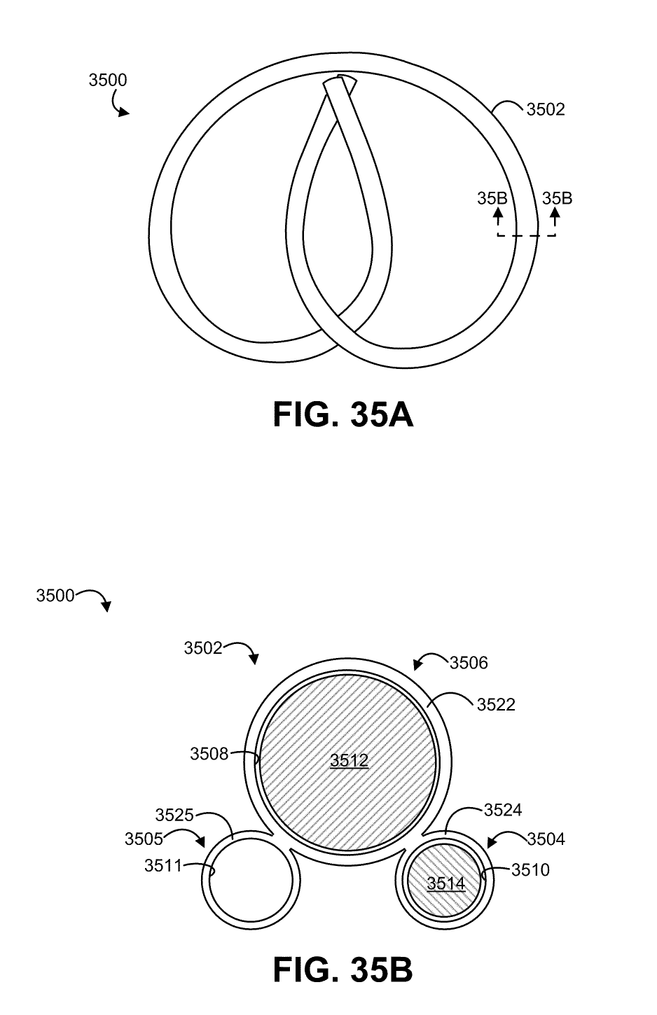

FIG. 35 illustrates an embodiment of an implantable device 3500 for passing over a guide wire, wherein FIG. 35A is a plan view of the implantable device 3500 and FIG. 35B is a cross-sectional view of the implantable device 3500. The implantable device 3500 may be similar to the device 100 described above with reference in FIG. 1, except that the device 3500 also may include a guide wire portion configured for passing the device 3500 over a guide wire.

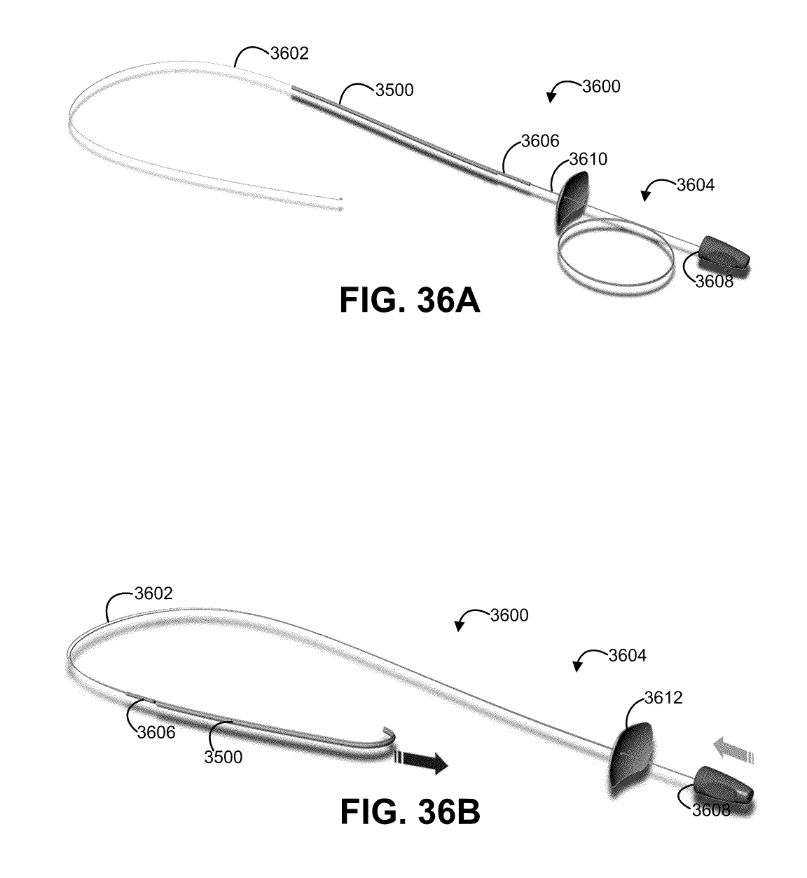

The device 3500 may be configurable between a relatively expanded shape suited for retention in the body, such as the shape shown in FIG. 35A, and a relatively lower-profile shape suited for deployment over a guide wire, such as the elongated shape shown in FIG. 36A. Following deployment into the body, the device 3500 may assume the relatively expanded shape to retain the device 3500 in the body cavity or lumen. As mentioned above, the relatively expanded shape may be any shape suited for retaining the device in the intended implantation location, including but not limited to the pretzel shape shown in FIG. 35A that is suited for retaining the device in the bladder, while the relatively lower-profile or deployment shape may be any shape suited for deploying the device 3500 into the body, including but not limited to the linear or elongated shape shown in FIG. 36. In embodiments, the device 3500 may naturally assume the relatively expanded shape and may be deformed, either manually or with the aid of an external apparatus, into the relatively lower-profile shape for insertion into the body. Once deployed the device 3500 may return to the initial, relatively expanded shape for retention in the body, such as spontaneously or naturally. However, the device 3500 may have other shapes and configurations that include a guide wire portion for deploying the device 3500 over a guide wire.

In the illustrated embodiment, the implantable device 3500 is a drug delivery device that includes a drug reservoir portion 3502, a retention frame portion 3504, and a guide wire portion 3505. The drug reservoir portion 3502 includes a drug reservoir lumen 3508 that houses a drug formulation 3512, such as a number of solid drug tablets. The retention frame portion 3504 includes a retention frame lumen 3510 that houses a retention frame 3514. The guide wire portion 3505 includes a guide wire lumen 3511 that houses an empty channel or bore so that a guide wire can be threaded therethrough.

The device 3500 generally includes a device body 3506. The device body 3506 includes a tube or wall 3522 that defines the drug reservoir lumen 3508, a tube or wall 3524 that defines the retention frame lumen 3510, and a tube or wall 3525 that defines the guide wire lumen 3511. The tubes or walls 3522, 3524, 3525 may be longitudinally aligned and coupled to each other along their length, and the lumens 3508, 3510, 3511 may be discrete from each other. Such a configuration may be achieved by integrally forming the device body 3506 via molding or extrusion. However, other configurations are possible. For example, the tubes 3522, 3524, 3525 may be separately constructed and assembled, either as shown or with two or more of the portions attached at discrete points but otherwise separate or spaced apart from each other. Also, the tubes 3522, 3524, 3525 and lumens 3508, 3510, 3511 may be substantially cylindrical, with the drug reservoir lumen 3508 having a relatively larger diameter than the retention frame lumen 3510 and guide wire lumen 3511 as shown, although other configurations can be selected based on, for example, the amount of drug to be delivered, the diameter of the retention frame, and deployment considerations such as the outer diameter of the guide wire.

The walls 3524, 3525 that define the retention frame lumen 3510 and the guide wire lumen 3511 may extend along the entire length of the wall 3522 that defines the drug reservoir lumen 3508, so that the retention frame lumen 3510 and the guide wire lumen 3511 have the same length as the drug reservoir lumen 3508 as shown, although one or more of the walls may be shorter than one or more of the other walls in other embodiments. Further, the walls 3522, 3524, 3525 are attached along the entire length of the device in the illustrated embodiment, although intermittent attachment can be employed.

The material used to form the device body 3506 may be elastic and/or flexible to permit moving the device 3500 between deployment and retention shapes. For example, the device body 3506 may be elastically deformed along with the drug 3512 and retention frame 3514 for implantation. The material used to form at least the wall 3522 about the drug reservoir lumen 3508 of the device body 3506 also may be water permeable or porous so that solubilizing fluid can enter the drug reservoir portion 3502 to solubilize the drug 3512 once the device is implanted. In preferred embodiments, the device body 3506 is formed from a material that is both elastomeric and water permeable. For example, silicone or another biocompatible elastomeric material may be used.

In one embodiment, the device body 3506 is non-resorbable. It may be formed of a medical grade silicone tubing, as known in the art. Other examples of suitable non-resorbable materials include synthetic polymers selected from poly(ethers), poly(acrylates), poly(methacrylates), poly(vinyl pyrolidones), poly(vinyl acetates), poly(urethanes), celluloses, cellulose acetates, poly(siloxanes), poly(ethylene), poly(tetrafluoroethylene) and other fluorinated polymers, poly(siloxanes), copolymers thereof, and combinations thereof

In some embodiments, the device body is bioerodible. In one embodiment of a bioerodible device, the tube of the body is formed of a biodegradable or bioresorbable polymer. Examples of suitable such materials include synthetic polymers selected from poly(amides), poly(esters), poly(ester amides), poly(anhydrides), poly(orthoesters), polyphosphazenes, pseudo poly(amino acids), poly(glycerol-sebacate)(PGS), copolymers thereof, and mixtures thereof. In a preferred embodiment, the resorbable synthetic polymers are selected from poly(lactic acids), poly(glycolic acids), poly(lactic-co-glycolic acids), poly(caprolactones), and mixtures thereof. Other curable bioresorbable elastomers include poly(caprolactone) (PC) derivatives, amino alcohol-based poly(ester amides) (PEA) and poly (octane-diol citrate) (POC). PC-based polymers may require additional cross-linking agents such as lysine diisocyanate or 2,2-bis(ε-caprolacton-4-yl)propane to obtain elastomeric properties. The different portions of the device body 3506 also may be formed from different materials in some embodiments.

The drug formulation 3512 may include essentially any therapeutic, prophylactic, or diagnostic agent, such as one that would be useful to deliver locally to a body cavity or lumen or regionally about the body cavity or lumen. In certain embodiments, the drug formulation 3512 is used to treat conditions such as interstitial cystitis; radiation cystitis; painful bladder syndrome; prostatitis; urethritis; post-surgical pain; neurogenic bladder; kidney stones; pain, urinary urgency or urinary frequency resulting from ureteral stent placement; urinary incontinence, frequency, or urgency, including urge incontinence and neurogenic incontinence; trigonitis; urinary tract cancer, such as bladder cancer and prostate cancer; pain, or any combination thereof. The drug formulation 3512 may include one or more local anesthetic agents, such as an aminoamide, an aminoester, or a cocaine analogue. The drug also can be an antimuscarinic compound, such as oxybutynin or propiverine. In particular embodiments, the drug formulation 3512 includes lidocaine.

In embodiments, the drug formulation 3512 is in the form of a number of drug units loaded in the drug reservoir lumen 3508 in a serial arrangement. For example, anywhere from between about 10 and about 100 drug units 3512 may be loaded therein, although any number of drug units may be used. The drug tablets 3512 may include lidocaine. In some embodiments, each drug tablet 3512 may be at least 70% lidocaine or more. Once the drug units 3512 are loaded in the drug reservoir lumen 3508, interstices or breaks may be formed between adjacent drug units 3512. The interstices or breaks may serve as reliefs that accommodate deformation or movement of the device 3500, while permitting the individual drug units 3512 to retain their solid form during storage and deployment. Thus, the drug delivery device 3500 may be relatively flexible or deformable despite being loaded with a solid drug, as each drug unit 3512 may be permitted to move with reference to adjacent drug units 3512. Along the length of the device drug reservoir lumen 3508, the drug units 3512 may have the same composition or may vary in composition, and in some cases drug units 3512 of different compositions may be in distinct reservoirs that are segregated, either axially or radially, along the length of the drug reservoir lumen 3508. The drug formulation 3512 also may have other forms, such as liquid or semi-solid forms.

The drug reservoir portion 3502 may release drug as described in other U.S. patent applications incorporated herein by reference. For example, the drug reservoir portion 3502 may operate as an osmotic pump. The tube 3522 may be formed from a water permeable material, such as a silicone. Following implantation, water or urine permeates through the wall 3522. The water enters the lumen 3508 and is imbibed by the drug formulation 3512. Solubilized drug is dispensed at a controlled rate out of the reservoir through one or more apertures in the wall 3522, driven by osmotic pressure in the reservoir. In an alternative embodiment, the device may operate essentially by diffusion of the drug through (i) one or more discrete apertures formed in the wall 3522, or passing pores formed in the wall 3522, or (ii) through the wall 3522 of the tube itself, which may be permeable to the drug, or (iii) a combination thereof. In embodiments in which diffusion occurs through the wall 3522, the apertures or passing pores may not be included. In still other embodiments, the device 3500 may operate by a combination of osmosis and diffusion.

In some embodiments, the drug reservoir portion 3502 may have multiple reservoirs or lumens, which permits segregating two or more different drug formulations 3512 in different reservoirs, delivering a single drug from different reservoirs at different rates or times following implantation, or combinations thereof. The two different reservoirs or lumens also may house the same or different drug formulations 3512 in the same or different forms (such as liquid, semi-solid, and solid), or combinations thereof. The two different reservoirs or lumens further may be configured to release drug via different release mechanisms, such as via osmosis through an aperture and by diffusion through a drug reservoir wall that may lack an aperture completely. Coatings or sheaths also may be provided along different portions of a single drug reservoir or along different drug reservoirs housing the same or different drug formulations. These embodiments can be combined and varied to achieve the desired release profile of the desired drug.

The retention frame lumen 3510 is loaded with the retention frame 3514, which may be an elastic wire. The retention frame 3510 may be configured to return to a retention shape, such as the illustrated “pretzel” shape or another coiled shape. In particular, the retention frame 3514 may retain the device 3500 in the body, such as in the bladder. For example, the retention frame 3514 may have an elastic limit and modulus that allows the device 3500 to be introduced into the body in a relatively lower-profile shape, permits the device 3500 to return the relatively expanded shape once inside the body, and impedes the device from assuming the relatively lower-profile shape within the body in response to expected forces, such as the hydrodynamic forces associated with contraction of the detrusor muscle and urination. Thus, the device 3500 may be retained in the body once implanted, limiting or prevent accidental expulsion.

The guide wire lumen 3511 may be substantially or completely empty. In particular, the guide wire lumen 3511 may have define a hollow bore with open ends. The bore may be sized and shaped for receiving a guide wire. For example, the bore may have a cross-sectional area or diameter that exceeds the cross-sectional area or diameter of the guide wire. The open ends of the lumen facilitate passing the guide wire therethrough. The device 3500 may be threaded over the guide wire by passing the guide wire through the guide wire lumen 3511. Although the guide wire lumen 3511 is described as receiving a guide wire, this empty third lumen 3511 may be employed for other purposes, such as for fluid delivery, as an electrode carrier, or in association with a deployment steering mechanism.

In some embodiments, the device 3500 may be deployed into the bladder using a specialized guide wire deployment system. FIG. 36 illustrates an embodiment of such a guide wire deployment system 3600 for deploying an implantable device into a bladder. The guide wire deployment system 3600 generally includes a guide wire 3602 and a plunger device 3604. The guide wire 3602 is longer than the urethra, such that when the guide wire 3602 is positioned in the urethra, its proximal end extends out from the urethra when its distal end is in the bladder. The guide wire 3602 is sized and shaped for fitting through the hollow bore of the implantable device 3500. For example, the guide wire 3602 may have a cross-sectional area or diameter that is less than the cross-sectional area or diameter of the hollow bore.

The plunger device 3604 includes a plunger 3606 operatively connected to a handle 3608 by way of a sheath 3610. The sheath 3610 is sufficiently rigid to transfer driving force from the handle 3608 to the plunger 3606. The plunger 3606 and the sheath 3610 have an internal bore suited for threading the plunger device 3604 over the guide wire 3602. Once the plunger device 3604 is so threaded, the handle 3608 is positioned on a proximal end of the guide wire 3602, the plunger 3606 is positioned on the guide wire 3602 rearward of its distal end, and the rigid sheath 3610 extends from the handle 3608 to the plunger 3606 so that a driving force applied to the handle 3608 can be transferred to the plunger 3606.

In use, the guide wire 3602 may be positioned in the urethra, such that its distal end is located in the bladder and its proximal end is exposed outside of the patient's body. The device 3500 may be threaded onto the guide wire 3602, with the guide wire 3602 passing through the guide wire lumen 3511. The plunger device 3604 may be threaded onto the guide wire 3602 and the handle 3608 may be advanced, so that the rigid sheath 3610 travels along the guide wire 3602 to advance the plunger 3606 until the device 3500 is pushed from the guide wire 3602. The guide wire 3602 then may be removed from the urethra, leaving the implantable device implanted in the bladder.

In some embodiments, the plunger device 3604 further includes a stop 3612. The stop 3612 is positioned at an appropriate position on the plunger device 3604 so that the stop 3612 contacts the body of the patient once the implantable device 3500 has separated from the guide wire 3602. Thus, a user is notified that the implantable device 3500 has been deployed in vivo, i.e. is implanted, so that the user can cease advancing the handle 3608 forward. In some embodiments, the stop 3612 may be adjustable to account for differences in anatomical sizes. The user may adjust the stop 3612 in advance of advancing the plunger device 3604, as needed.

The guide wire 3602 may facilitate straightening the implantable device 3500 from the retention shape, which is suited for retaining the device 3500 in the bladder, into the deployment shape, which is suited for passing the device 3500 through the urethra. In some embodiments, the guide wire 3602 may be used in association with a separate deployment catheter. In such embodiments, the guide wire 3602 may be threaded through the deployment catheter, although other configurations are possible. The guide wire 3602 may also have a J-shape or a curved shape suited for deploying an implantable device through the male urethra. The guide wire deployment system 3600 and implantable device 3500 also can be provided together in a package.

In some embodiments, any of the deployment instruments described above with reference to FIGS. 6-36 may be provided as part of a kit. The kit may include at least one package that houses one or more of the instrument, a stylet, a syringe, and the implantable device. The package protects the packaged components before the implantation procedure. For example, the components may be sterilized together, and transported together, in the package. In such embodiments, the implantable device can be pre-loaded into or onto the deployment instrument before the deployment instrument is placed in the package, which eliminates the need to load the implantable device during the implantation procedure, reducing the number of steps in the procedure and reducing the risk that the implantable device will be dropped inadvertently or damaged during loading. However, the implantable device need not be pre-loaded. Instead, the implantable device can be provided along side the deployment instrument or can be packaged separately. Also, a stylet can be either be packaged with the deployment instrument, packaged separately, or omitted completely. Regardless of which components are packaged together, the package is sterilized, such as using gamma irradiation or ethylene oxide sterilization.

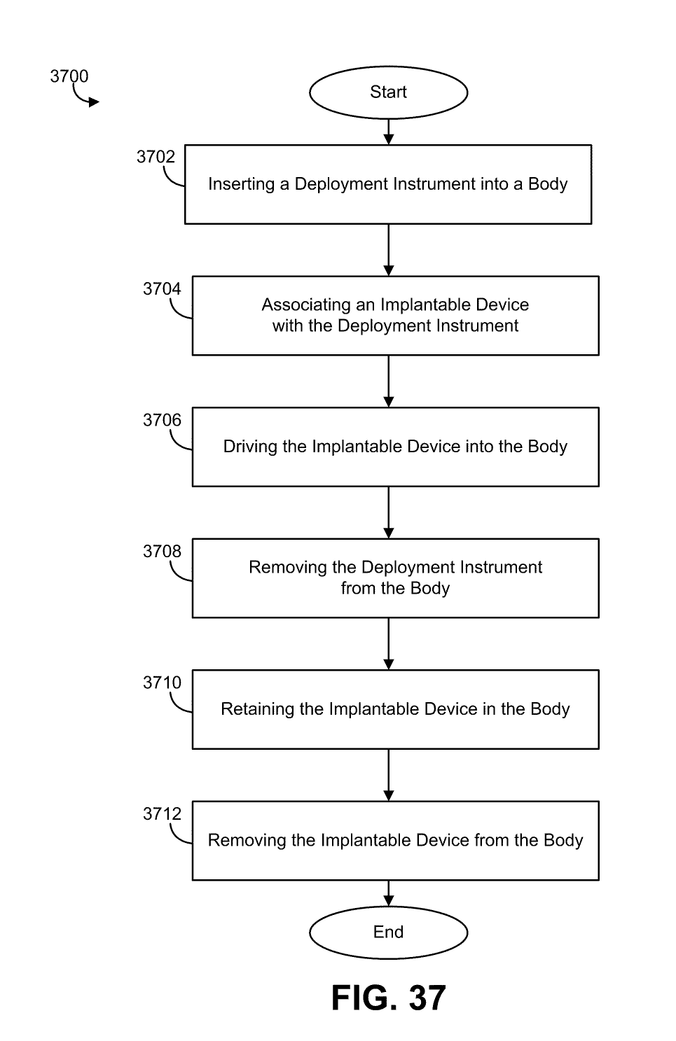

FIG. 37 is a block diagram illustrating an embodiment of a method 3700 for implanting an implantable device into the bladder, such as an implantable drug delivery device. In block 3702, a deployment instrument is inserted into the body. The deployment instrument may be an embodiment of the deployment instrument described above, although other deployment instruments may be used. For example, the deployment instrument may be a catheter or a cystoscope, either conventionally known or specially developed for this purpose. The deployment instrument also may be a guide wire. Inserting the deployment instrument generally includes inserting the deployment instrument into the urethra and driving the deployment instrument forward until a distal end is positioned in the bladder, while a proximal end remains outside of the body.

In some embodiments, the deployment instrument is inserted into the body in block 3702 in association with a cystoscope, which permits visualizing the implantation procedure. The cystoscope may be rigid or flexible and may include an interior channel. A rigid cystoscope may be preferred for female patients, while a flexible cystoscope may be preferred for male patients. In use, the cystoscope is inserted through the urethra and the deployment instrument is inserted through the interior channel of the cystoscope until the distal end of the deployment instrument reaches the bladder. In such embodiments, the deployment instrument may not need to have a mechanical stiffness or column strength suited for insertion through the urethra directly.

In some embodiments, inserting the deployment instrument into the body in block 3702 further includes verifying a distal end of the deployment instrument has become positioned in the bladder. For example, the location of the distal end can be verified by communicating urine through the deployment instrument from the distal end positioned in the bladder. The location of the distal end also can be verified by visualizing the distal end of the deployment instrument with a cystoscope, an ultrasound or x-ray. Also in some embodiments, inserting the deployment instrument into the body in block 3702 further includes securing a distal end of the deployment instrument in the bladder, such as by inflating a balloon positioned on the distal end.

In block 3704, the implantable device is associated with the deployment instrument. The implantable device can be any suitable device, including embodiments of the device 100 or the device 3500 described above. In some cases, associating the implantable device with the deployment instrument includes inserting the implantable device into a deployment instrument. Inserting the implantable device into the deployment instrument can include deforming the implantable device from a retention shape to a deployment shape. Inserting the implantable device into the deployment instrument may include inserting the implantable device into the deployment instrument distal end, into the deployment instrument proximal end, or into an opening or slit along the deployment instrument central body portion. In some cases, inserting the implantable device into the deployment instrument comprises pre-loading the implantable device into the deployment instrument before the deployment instrument is inserted into the body. In such cases, the order of blocks 3702 and 3704 is reversed. The implantable device is typically pre-loaded if the device is placed in the deployment instrument through the distal end or central body portion, although the implantable device can be pre-loaded in any case for convenience, such as in cases in which the components are provided as part of a kit. The implantable device may be lubricated before the implantable device is inserted into the deployment instrument, to reduce friction associated with driving the implantable device through the deployment instrument in block 3706. In cases in which the deployment instrument is a guide wire, associating the implantable device with the deployment instrument may include positioning the implantable device on the guide wire, such as by threading the guide wire through an opening or bore in the implantable device. The action of threading the implantable device onto the guide wire may cause the implantable device to assume an elongated deployment shape.

In block 3706, the implantable device is driven into the bladder. In embodiments in which the implantable device is inserted into the deployment instrument proximal end portion in block 3704, driving the implantable device into the bladder includes driving the implantable device from the deployment instrument proximal end portion, along the deployment instrument central body portion, and through the deployment instrument distal end portion until the implantable device exits the deployment instrument into the bladder. In embodiments in which the implantable device is inserted into the deployment instrument distal end in block 3704, driving the implantable device into the bladder includes driving the implantable device through the deployment instrument distal end portion until the implantable device exits the deployment instrument into the bladder, as the implantable device was previously driven through the urethra upon insertion of the deployment instrument in block 3702.

In some embodiments, driving the implantable device into the bladder in block 3706 comprises driving the implantable device with a stylet. The stylet may be an embodiment of the stylet described above. In such cases, driving the implantable device into the bladder in block 3706 includes inserting the stylet distal end portion into the deployment instrument proximal end portion and advancing the stylet through the deployment instrument until the stylet distal end portion reaches the deployment instrument distal end portion. In embodiments in which the implantable device is loaded into the deployment instrument proximal end portion, driving the implantable device into the bladder in block 3706 includes contacting the implantable device with the stylet, advancing the implantable device through the deployment instrument using the stylet, and driving the implantable device from the deployment instrument with the stylet. In embodiments in which the implantable device is preloaded into the deployment instrument distal end portion, driving the implantable device into the bladder in block 3706 includes advancing the stylet through the deployment instrument to the implantable device located at the deployment instrument distal end portion, contacting the implantable device with the stylet, and driving the implantable device from the deployment instrument with the stylet.

In other embodiments, driving the implantable device into the bladder in block 3706 comprises driving a stream of fluid (i.e., a liquid) into the deployment instrument. The stream of fluid may be a stream of incompressible lubricant, as described above. In such embodiments, driving the implantable device into the bladder may further comprise associating the proximal end of the deployment instrument with a fluid source such that a fluid-tight seal is formed. For example, a luer connector on the deployment instrument proximal end may be attached to a source of lubricant. The lubricant may be driven from the source of lubricant through the deployment instrument, pushing the implantable device forward until the device exits into the bladder. Thereafter, the lubricant may be naturally flushed from the body.

In still other embodiments, driving the implantable device into the bladder in block 3706 includes driving the device over a guide wire, such as with a pusher. The pusher may contact the implantable device, which may be mounted on the guide wire, and as the pusher is advanced forward the implantable device may ride along the guide wire. When the implantable device reaches the end of the guide wire, the implantable device is released into the body. For example, the end of the guide wire may be positioned in the bladder, in which case the device may be released into the bladder.

In embodiments in which the method implants a device that is configurable between a deployment shape and a retention shape, driving the implantable device from the deployment instrument in block 3706 removes the force of the deployment instrument wall from the implantable device, which may cause the implantable device to naturally return to the retention shape for retention in the bladder. An example is shown in FIG. 4 with reference to the implantable device of FIG. 1.

In some embodiments, driving the implantable device into the bladder in block 3706 includes observing the implantable device in the bladder to ensure the device was properly implanted. For example, the device may be observed using a cystoscope, ultrasound, or x-ray.

In some embodiments, driving the implantable device into the bladder in block 3706 further includes introducing a volume of fluid into the bladder before the device is driven into the bladder. The fluid may be, for example, water or saline. The fluid may serve as a cushion in the bladder, reducing the likelihood of the implantable device contacting the posterior of the bladder upon implantation, which may cause discomfort or bladder perforation.

In block 3708, the deployment instrument is removed from the body. If a stylet was employed in block 3706 to drive the implantable device into the bladder, the stylet is also removed. The stylet can be removed either before or simultaneously with the deployment instrument. Thereafter, in block 3710, the implantable device remains implanted in the body for a period, such as a period of hours, days, weeks, or even months. In embodiments in which the implanted device is a drug delivery device, the device remains implanted in the body in block 3710 to release a drug into the body, such as to release a drug from the device into the bladder and to the urothelial tissues and other local or regional tissues.

In some embodiments, the method includes removing the implantable device from the body in block 3712. Removing the device from the body may include inserting a removal instrument into the body, locating the implanted device in the body, and pulling the implanted device from the body through the removal instrument. For example, the removal instrument may be a cystoscope or catheter that is inserted through the urethra until its distal end reaches the bladder.

Once the removal instrument is positioned in the body, the implanted device may be located in the body and is pulled into the removal instrument. For example, forceps passed through the removal instrument may be used to locate the implanted device and to pull the device into the removal instrument. The device may fold upon itself or assume the deployment shape as it enters the removal instrument. In embodiments in which the device is an implantable drug delivery device, a drug reservoir portion of the device may be at least partially depleted or empty upon removal, facilitating removal. In some embodiments, the device may be located and pulled into the removal instrument using a retrieval feature. For example, the device may include a magnetized portion that is drawn to a magnetized portion of the removal instrument and/or a magnetized portion of the forceps, facilitating retrieval. In other embodiments, the implantable device may include a string that is grasped to pull the implantable device into the removal instrument.

Once the implantable device is located, the implantable device may be pulled through the removal instrument, and thereafter the removal instrument may be removed from the body. Alternatively, the implantable device and removal instrument may be removed from the body simultaneously.

Embodiments for retrieval features for implantable devices are also disclosed herein. The retrieval feature facilitates removing the implantable device from the body, such as after the drug has been released. One example of a retrieval feature is a string. The string may be attached to a mid-portion or an end portion of the implantable device. In some embodiments, the string is sized to extend from the bladder through the urethra to an exterior of the body, in which case once the implantable device is positioned in the bladder, a proximal end of the string may be positioned outside of the body. Thus, the string may be engaged to pull the implantable device from the bladder through the urethra. The string also may be shorter in size, so that once the implantable device is positioned in the bladder, the proximal end of the string is positioned in the urethra in a location that is reachable by a physician. In either case, the implantable device may be removed from the bladder by engaging the string to pull the implantable device through the urethra. In such embodiments, the diameter of the string may be sized to fit comfortably in the urethra during the period of implantation. In other embodiments, the string is sized to be wholly implanted in the bladder with the implantable device, in which case the string facilitates locating and grasping the implantable device within the bladder using a removal instrument positioned in the urethra, such as forceps with the aid of a cystoscope or catheter.

In embodiments in which the string is attached to a mid-portion of the drug delivery device, the device may fold upon itself as it enters the removal instrument or the urethra. For example, an implantable device that releases drug in vivo may be at least partially empty upon removal, facilitating folding of the device along its mid-portion for passage through the removal instrument or the urethra. In embodiments in which the string is attached to an end-portion of the implantable device, the implantable device may move into the deployment shape as it enters the removal instrument or the urethra. Thus, the deployment shape also may be considered a retrieval shape in such embodiments. The string also may be used during the implantation procedure to identify when the implantable device has become implanted. As the implantable device travels along the urethra toward the bladder, the implantable device may pull the string forward. Once the implantable device has become implanted, the string may come close to entering the body or any deployment instrument through which the implantable device is inserted.

In other embodiments, the implantable device is associated with a string that facilitates loading the implantable device into a deployment instrument. The string may be at least as long as the deployment instrument. The string may have a fixed end attached to the implantable device and a free end opposite from the fixed end. To load the implantable device, the free end of the string may be threaded through one end of the deployment instrument and along the length of the deployment instrument until it emerges from the opposite end. The string is then pulled to pull the implantable device into the one end of the deployment instrument and along its length until the implantable device nears the opposite end. Thereby, the implantable device may be loaded into the deployment instrument. Before the deployment instrument is inserted into the body, the string may be cut near the attached end to remove the string from the implantable device.

Another example of a retrieval feature is a magnet. For example, the implantable device may include a magnetized portion, such as a mid-portion or end portion. The magnetized portion is configured to draw the implantable device toward a magnetized removal instrument. For example, the magnetized removal instrument may be a cystoscope or a catheter having a magnetized distal end. The magnetized removal instrument may be inserted through the urethra until its magnetized distal end becomes positioned into the bladder. The magnetized distal end may draw the magnetized portion of the implantable device toward it so that the implantable device can be pulled into the removal instrument, such as with a pair of forceps. In other embodiments, the forceps may be magnetized, either as an alternative to or in addition to the magnetized distal end of the removal instrument.

In some embodiments, the deployment instrument is associated with a retention feature. The retention feature may be used to maintain or anchor a portion of the deployment instrument, such as its distal end, in the bladder. For example, the deployment instrument may have a retention feature located at its distal end portion. The retention feature may be movable between a deployment shape or configuration and a deployed shape or configuration. In the deployment configuration, the retention feature assumes a lower profile for deployment through the urethra into the bladder. In the deployed configuration, the retention feature assumes a larger profile for retaining the distal end in the bladder. In some cases, the retention feature may assume the deployed configuration upon exiting the urethra into the bladder, while in other cases user intervention may be required.

One example of such a deployment instrument is the embodiment shown in FIG. 30, which includes an inflatable balloon at its distal end. The balloon can be inflated to retain the distal end in the bladder. Another example of a retaining feature for a deployment instrument is a number of retaining arms. The retaining arms may be attached to a distal end of the deployment instrument. The retaining arms may extend away from the distal end of the deployment instrument and may terminate in a free end. The retaining arms may be loaded such that the free end is naturally biased away from the deployment instrument. As the deployment instrument is inserted through the urethra, the urethra exerts a force against the retaining arms to maintain the arms inward. Once the distal tip of the deployment instrument is far enough into the bladder so that the free ends of the retaining arms are exposed, the retaining arms return outward to retain the distal tip of the deployment instrument in the bladder. The retaining arms increase the force needed to remove the distal tip of the deployment instrument from the bladder, but the retaining arms do not prevent removal of the deployment instrument. Thus, once the implantation procedure is completed, the deployment instrument can be removed from the bladder by applying increased pressure to bend the retaining arms upward as the deployment instrument is pulled through the urethra.

In some embodiments, a collar or secondary catheter is positioned about the deployment instrument to maintain the arms in a closed position as the deployment instrument is inserted. Once the distal tip of the deployment instrument emerges into the bladder, the collar or secondary catheter is pulled backward, causing the retaining arms to return to an expanded configuration. The retaining arms increase the force needed to remove the distal tip of the deployment instrument from the bladder. Thus, the deployment instrument can be removed from the bladder by applying increased pressure that bends the retaining arms inward as the deployment instrument is pulled through the urethra. Alternatively, the collar or secondary catheter can be extended about the retaining arms to draw the arms inward before the deployment instrument is removed from the urethra.

In other embodiments, the deployment instrument includes a movable distal tip. The movable distal tip is associated with the wall of the deployment instrument via at least two movable arms. The movable distal tip also is associated with a string that extends through the length of the deployment instrument to its proximal end. Once the distal tip of the deployment instrument is positioned in the bladder, the string is pulled to move the distal tip toward the deployment instrument, causing the retaining arms to bend outward. The retaining arms may increase the force needed to remove the distal tip of the deployment instrument from the bladder.

To remove the deployment instrument, the string is released to permit the arms to return as the deployment instrument is pulled through the urethra.

In other embodiments, the deployment instrument has a coiled distal end portion. The coiled distal end portion may be moved or deformed into an elongated configuration for insertion through the urethra but may naturally return to a coiled configuration once the deforming force of the urethra walls is removed. Alternatively, wire may be inserted through the deployment instrument to cause the coiled distal end portion to assume an elongated configuration for insertion, and the wire can be removed after insertion. The coiled distal end portion may facilitate retaining the deployment device in the bladder. The coiled distal end portion also may facilitate delivering the implantable device farther in the bladder, reducing the likelihood of the device contacting the sensitive trigone region near the bladder neck upon deployment. The coiled distal end portion increases the force needed to remove the distal end portion of the deployment instrument from the bladder, and thus the deployment instrument can be removed by pulling with increased force.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. An implantable drug delivery device comprising:
a drug reservoir portion housing a drug formulation;
a retention frame portion housing a retention frame; and
a guide wire portion defining a hollow bore for receiving a guide wire.

2. The implantable drug delivery device of claim 1, wherein the drug formulation comprises a plurality of drug tablets.

3. The implantable drug delivery device of claim 2, wherein at least some of the drug tablets comprise lidocaine.

4. The implantable drug delivery device of claim 1, wherein the retention frame comprises an elastic wire.

5. The implantable drug delivery device of claim 1, wherein the retention frame is configured to assume a pretzel-like shape.

6. The implantable drug delivery device of claim 1, wherein the device is deformable from a retention shape to an elongated shape.

7. An implantable drug delivery device comprising:
a device body that includes a first wall defining a drug reservoir lumen, a second wall defining a retention frame lumen, and a third wall defining a guide wire lumen;
a plurality of solid drug tablets housed within the drug reservoir lumen;
a retention frame housed within the retention frame lumen; and
a hollow bore within the guide wire lumen.

8. The implantable drug delivery device of claim 7, wherein the device body is deformable.

9. The implantable drug delivery device of claim 7, wherein at least some of the plurality of solid drug tablets comprise lidocaine.

10. The implantable drug delivery device of claim 7, further comprising an interstice formed between any two adjacent drug tablets, the interstice facilitating deformation of the device body.

11. The implantable drug delivery device of claim 7, wherein the device body is integrally formed by an extrusion process or a molding process.

12. The implantable drug delivery device of claim 7, wherein the first wall comprises a water permeable silicone.

13. The implantable drug delivery device of claim 7, wherein the retention frame is configured to assume a pretzel-like shape.

14. The implantable drug delivery device of claim 7, wherein the retention frame comprises an elastic wire.

15. The implantable drug delivery device of claim 7, wherein the hollow bore is sized and shaped for receiving a guide wire.

16. A system comprising:
an implantable drug delivery device comprising:
a device body that includes a first wall defining a drug reservoir lumen, a second wall defining a retention frame lumen, and a third wall defining a guide wire lumen,
a plurality of solid drug tablets housed within the drug reservoir lumen,
a retention frame housed within the retention frame lumen,
a hollow bore within the guide wire lumen;
a guide wire deployment system for implanting the implantable drug delivery device, the guide wire deployment system including:
a guide wire, and
a plunger device for pushing the implantable drug delivery device over the guide wire.

17. The system of claim 16, wherein the guide wire is longer than the urethra, such that when the guide wire is positioned in the urethra with its distal end in the bladder, its proximal end extends out from the urethra.

18. The system of claim 16, wherein the guide wire has a cross-sectional area that is sized and shaped for fitting through the hollow bore of the implantable drug delivery device.

19. The system of claim 16, wherein the plunger device comprises:
a plunger;
a handle;
a sheath extending between the plunger and the handle, the sheath transferring to the plunger a driving force applied to the handle; and
an internal bore for receiving the guide wire, such that the plunger device can travel over the guide wire.

20. The system of claim 16, wherein the pusher further comprises a stop configured to indicate that the implantable drug delivery device has separated from the guide wire.
